(19) United States
(12) Patent Application Publication
(10) Pub. No.: US 2007/0156082 A1
(43) Pub. Date: Jul. 5, 2007

(54) DEVICE FOR TRANSFERRING MOLECULES TO CELLS USING AN ELECTRIC FORCE

(76) Inventor: Yves Scherman, Villejuif (FR)

Correspondence Address: Judy Jarecki-Black, Ph.D, Esq.;Merial Limited, 3239 Satellite Blvd, Duluth, GA, 30096, US

(21) Appl. No.: 11/615,991

(22) Filed: Dec. 24, 2006

Related U.S. Application Data
(63) Continuation-in-part of application No. PCT/FR05/01611, filed on Jan. 24, 2005.

(30) Foreign Application Priority Data
Jun. 24, 2004 (FR) 0406943
Sep. 27, 2004 (FR) 0410172
Jan. 20, 2005 (FR) 0500603
Mar. 4, 2005 (FR) 0502215

(51) Int. Cl. A61N 1/30

(52) U.S. Cl. 604/20

(57) ABSTRACT

The invention concerns a device and a method for optimal delivery of an active principle (36) into a human or animal tissue for chemotherapy or gene therapy, using an electric field or current. The device consists of electrodes connected to an electric current generator, providing better efficiency, reproducibility and safety which are achieved through the use of adapted electrode devices and use of optimal current intensity.

### REFERENCE TO RELATED APPLICATIONS

This application is a continuation-in-part of International Patent Application No. PCT/FR2005/001611 filed Jun. 24, 2005 and published as WO 2006/010837 on Feb. 2, 2006, which claims priority to French Application Nos. 0406943 filed Jun. 24, 2004, 0410172 filed Sep. 27, 2004, 0500603 filed Jan. 20, 2005 and 0502215 filed Mar. 4, 2005.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention relates to a device allowing the administration of substances, including active ingredients, into tissues in vivo and into the cells of these tissues, whereby the administration is improved by combining the injection of active ingredient with a physical method comprising the administration of electric fields and/or electric currents. These fields and/or this current have the effect of temporarily permeabilizing the cells and also of improving the penetration of the active ingredient into the tissue.

### BACKGROUND

Electrotransfer or electropermeabilization involves the injection of a chemical molecule or a nucleotide into a tissue, and the simultaneous or subsequent administration of pulses of fields and electric current, which permeabilize the wall of the cells and thereby promote the entry of the nucleotide into the cell and, in some cases, into the nucleus.

This improved administration results from an electrophoresis or iontophoresis effect on the active ingredient, by which the molecules of the active ingredient are entrained by electric convection into the tissues or actually within human, animal, plant or bacterial cells.

In the present application, “electrotransfer” refers to this method of electrically assisted administration, using the electric fields and/or electric currents to improve the administration of an active ingredient to biological tissues, as well as its effectiveness. “Fields” refers to the electric and electromagnetic fields, as well as the electric current, which are delivered between two electrodes subjected to different voltages.

Antitumoral chemotherapy is an example in which the penetration of chemical molecules inside tumoral cells is necessary for a therapeutic activity according to the “electrochemotherapy” method.

The effect of the electrotransfer of DNA is to promote the tissular and intracellular penetration of these DNAs, which makes possible their expression in the form of RNA or of protein if these DNAs contain a gene preceded by a promoter and followed by a polyadenylation sequence (this is called a therapeutic expression cassette).

In the present application, “active ingredient” refers to any molecule having a beneficial effect or for analytical purposes such as imagery, functional, and in particular, all macromolecules of peptide or nucleic acid type. Of the nucleic acids, plasmids or linear DNA or RNA strands produced by synthesis are a preferred form. The invention also relates to any nucleic acid, any protein, any sugar or any other genetically or chemically modified biological molecule, or also any wholly synthetic molecule, produced according to the methods of a person skilled in the art.

Electrotransfer can be used in particular for coding DNAs, plasmids or any other form of genetic material (DNA, RNA or other) leading to the expression of the product of this gene, this product being an RNA or a protein, including, but not limited to, tissues such as muscles, tumors, skin, nervous system or liver.

Electrotransfer requires a device composed of as a minimum, a generator of electric pulses and a device with electrodes.

U.S. Pat. No. 5,273,525 describes a syringe comprising two injection needles which also serve as electrodes.

U.S. Pat. No. 6,055,453 describes the use of a set of identified electrodes allowing selection of a sequence of application of electric fields.

WO patent 03/086534 presents a system with two needle electrodes and an injection needle in the centre.

WO patent 02/098501 describes a non-ordered group of needle electrodes, one of them able to allow the injection of the active ingredient at the surface of the skin.

US patent 2004/0092860 proposes a set of several needle electrodes used for injection, an injection needle being located in the centre.

U.S. Pat. No. 5,318,514 describes devices with electrodes.

US patent 98/08183 describes groups of configurations of networks of electrodes.

US patent 2005/070841 describes a device comprising two injection needles which also serve as electrodes.

US patent 2005/070841 describes a device comprising two injection needles which also serve as electrodes.

US patent 2003/0009148 describes electrodes in the form of invasive needles allowing the integration of a tubular electrode in its centre.

Finally, some types of generators of electric pulses are already manufactured and marketed.

The citation or identification of any document in this application is not an admission that such document is available as prior art to the present invention.

### SUMMARY OF THE INVENTION

The electrotransfer of DNA involves the delivery of electric pulses with a voltage difference that can be more than 30 V over periods that may be of the order of several tens of milliseconds. This causes serious, even intolerable, pain and muscular contractions that may have serious, sometimes irreversible, consequences. Muscular contraction can occur even if the subject is anesthetized. Moreover, in numerous cases, the weak state or the age of the subject means that anesthesia is contra-indicated.

The present invention proposes devices with invasive and non-invasive electrodes and methods allowing the efficiency and the harmlessness of the electrotransfer to be improved and its toxicity reduced, and the extent of the muscular contractions reduced.

The present invention also allows efficiency to be improved by optimizing the position of the electrodes and the choice of the fields delivered. By “deliver” or “delivery” of the fields is meant in the present application the generation by the generator of a potential difference between the electrodes, thus generating an electric current and/or electric fields between the electrodes. The enhancement of efficiency creates scope, allowing if necessary the reduction in harmfulness by reducing the parameters causing pain and muscular contraction. The present invention proposes a casing device allowing the device with electrodes to be gripped and the latter to be electrically connected to the generator.

In the present application, by “a set of electrodes” is meant a group of at least 1 electrode.

The present invention proposes the following devices and methods.

The present invention is composed of a device for improving the in vivo penetration of molecules of active ingredient into the cells of the tissues of a human or animal subject, characterized in that it comprises:
a generator of electric pulses,

a first group of electrodes composed of at least one electrode electrically connected to a first terminal of the generator of electric pulses,

a second group of electrodes composed of at least one electrode electrically connected to the second terminal of the generator of electric pulses, and

a means of injecting the active ingredient into the tissues.

In one embodiment of the invention, the first group of electrodes is composed of a single electrode.

In another embodiment of the invention, the electrode is invasive.

In yet another embodiment of the invention, the electrode has a means of injecting the active ingredient.

It is also an embodiment of the present invention that the electrode is an injection needle.

In one embodiment of the invention, a means of injecting the active ingredient is constituted by at least one integral needle and placed close to the electrode and at a depth less than the depth of the electrode.

A further embodiment of the invention is that:
the electrode is also constituted by a catheter envelope pierced by orifices of sufficient size to permit a contact between the needle and the tissues through these orifices; the invasive needle of the electrode passes through the catheter along its axis and can slide along the axis of the catheter and can be withdrawn from the catheter; and,
the catheter and the needle, once assembled, form an invasive electrode, these electrodes being hereinafter called “electrode partly covered by a non-conductive catheter”.

In yet a further embodiment of the invention, the electrode is constituted by:
a catheter, covered by an electrically conductive surface connected to the corresponding terminal of the pulse generator, hereinafter called “conductive catheter electrode”, its surface being composed of a material approximately preserving the flexibility of the catheter, which catheter can serve as means of injecting the active ingredient; and,
an invasive needle passing through the catheter along its axis and allowing penetration of the tissues and able to slide along the axis of the catheter and able to be withdrawn from the catheter, which needle can also serve as injection means.

Another embodiment of the present invention includes the invasive electrode being covered, in its upper part penetrating into the tissues, by an electric insulating material.

In a further embodiment, at least one catheter electrode is magnetized.

In yet another embodiment of the present invention, the second group of electrodes comprises at least one non-invasive electrode arranged on the surface of the tissues covering the zone containing the active ingredient.

An additional embodiment of the present invention is that at least one non-invasive electrode has an orifice allowing the invasive electrode to pass through it.

In a still further embodiment, the present invention also comprises a guide allowing the axis of the invasive electrode to be directed in a predefined direction.

An embodiment of the present invention is that the second group of electrodes comprises a single invasive electrode.

In another embodiment, the electrode of the second group is an electrode partly covered by a non-conductive catheter.

A further embodiment of the present invention is that the electrode of the second group is constituted by a conductive catheter electrode.

Yet a further embodiment of the present invention is that the electrode of the first group is a catheter electrode and, where the two needles are parallel, integral and connected by a support.

In another embodiment, the two invasive electrodes are integral, assembled with the help of a support, approximately parallel and approximately of the same depth.

It is also within the scope of the invention that the second group of electrodes comprises a plurality of invasive electrodes.

In one embodiment, the invasive electrodes of the second group are situated approximately on a circle of which the electrode of the first group approximately forms the centre.

In another embodiment, the invasive electrodes of the second group border the zone where the active ingredient is injected.

A further embodiment of the present invention is that two invasive electrodes of the second group of electrodes are substantially aligned with the electrode of the first group.

In yet a further embodiment, the invention also comprises a means allowing orientation of the electrodes along a parallel axis.

Another embodiment of the present invention is that all the electrodes are integral, assembled with the help of a support, approximately parallel and approximately of the same depth.

It is within the scope of the present invention that the device also comprises at least one non-invasive electrode connected to one of the terminals of the generator.

Furthermore, the present invention can comprise a device wherein:
each invasive electrode can have a casing allowing a good grip on the electrode and ensuring the electric connection between the electrode and its terminal of the generator
the casing having a housing allowing the electrodes and the means of injecting the active ingredient and the reservoir containing the active ingredient to be accommodated.

Also within the scope of the present invention is a device wherein:
a plurality of electrodes have a single casing allowing a good grip on the electrode device and ensuring the electric connection between each electrode and its terminal of the respective generator
the casing has a housing allowing the electrodes and the means of injecting the active ingredient and the reservoir containing the active ingredient to be accommodated.

In one embodiment of the present invention, the casing also comprises the means of orienting the electrodes along a parallel axis.

In another embodiment of the present invention:
the integral electrodes and their support have a casing allowing a good grip on the electrode device and ensuring the electric connection between each electrode and its terminal of the generator; and

the casing has a housing allowing the integral electrodes and their support and the means of injecting the active ingredient and the reservoir containing the active ingredient to be accommodated.

In yet another embodiment of the present invention the casing:
has a means allowing the electrode to be successively pushed into the tissues to predefined intermediate depths;
and has a means allowing the injection of the active ingredient at each stop position.

In a further embodiment of the present invention, the means allowing the electrode to be successively pushed into the tissues to predefined intermediate depths is constituted by at least one stop.

In yet a further embodiment of the present invention, a plurality of invasive electrodes connected to the generator are covered, in their upper part penetrating into the tissues, by an electric insulating material.

It is additionally within the scope of the invention that all the invasive electrodes connected to the generator are covered, in their upper part penetrating into the tissues, by an electric insulating material.

In one embodiment of the present invention, the two groups of electrodes comprise a set of non-invasive electrodes arranged on the surface of the tissues covering the zone containing the active ingredient.

In yet a further embodiment, the electrodes are applied to a single face of the tissues containing the active ingredient so as permit the delivery of electric fields spreading under the surface of the tissues to which the electrodes are applied.

In another embodiment, the surface in contact with the tissues of at least one non-invasive electrode has an approximately rectangular shape.

In an additional embodiment, the surface in contact with the tissues of at least one non-invasive electrode has a shape approximately resembling a horseshoe.

It is also an embodiment of the invention that the surface of at least one non-invasive electrode is flexible.

In another embodiment of the present invention, a non-invasive electrode has the shape of a tip flattened at its end.

In yet another embodiment of the present invention, the non-invasive electrodes form an integral part of an elastic sheath.

It is also an embodiment of the present invention that at least one non-invasive electrode is wire-shaped.

In a further embodiment, the device comprises only 2 wire-shaped non-invasive electrodes, these electrodes following a different axis.

In yet a further embodiment of the present invention, the surface in contact with the tissues of at least one non-invasive electrode has an irregular shape.

It is also an embodiment of the present invention that the non-invasive electrodes are asymmetrical.

It is within the scope of the present invention that at least one non-invasive electrode is composed of several non-invasive electrodes electrically connected to one another.

The methods of the present invention use a device having the embodiments described above. This method allows the penetration of molecules of active ingredient in vivo into the cells of the tissues of a human or animal subject to be improved; this method comprising the following stages:
at least one electrode electrically connected to the first terminal of a pulse generator and at least one electrode electrically connected to the second terminal of the pulse generator is put into contact with the tissues to be treated and the active ingredient is injected into the zone of tissues to be treated; and,

the emission of electric pulses by the generator is then triggered, the amplitude of the electric signals being calculated as a function of the distance between the electrodes and the nature of the tissues, so as to create an electric field between the electrodes, this allowing the active ingredient to penetrate into the tissues and into the cells.

It is within the scope of the method of the present invention that the active ingredient is injected into a sealed cavity which contains the tissues to be treated and containing a fluid material.

In one embodiment of the method of the active ingredient is injected into the synovial cavity.

In yet another embodiment of the method of the present invention, before delivering the fields:
a set of invasive electrodes connected to the two terminals of the generator is successively pushed into the tissues to intermediate depths
the active ingredient is injected into the tissue to successive depths using said electrodes,

all the invasive electrodes are then pushed in to a predefined final depth before triggering the delivery of the electric fields.

A further embodiment of the method of the present invention is that, before delivering the fields:
the invasive electrodes connected to the first terminal of the generator are introduced to the same depth into the tissues along the same axis and in the centre of the zone containing the active ingredient; and

the invasive electrodes connected to the second terminal of the generator are introduced into the tissues, along the same axes and to substantially to the same depth as the invasive electrodes connected to the first terminal of the generator, approximately bordering the zone of tissues containing the active ingredient, the electrodes being positioned at an approximately identical distance from the centre of the zone containing the active ingredient and being distributed regularly around this centre.

Yet a further embodiment of the method of the present invention is that the active ingredient is injected using the electrodes connected to the first terminal of the generator.

It is within the scope of the present invention that, before delivering the fields:
at least one catheter electrode is connected to a terminal of a generator;
at least one non-invasive electrode is connected to the other terminal of the generator;
each catheter electrode is pushed into the tissues in order to penetrate into the cavity; the needle is then slid inside each catheter so as not to damage the walls of the cavity during the shifting of the catheter electrode inside the cavity and during the delivery of the fields;
the active ingredient is injected using at least one catheter electrode; and,

each non-invasive electrode is placed at the edge of the cavity.

It is also within the scope of the method of the present invention that at least one catheter electrode is connected to one terminal of a generator, at least one catheter electrode is connected to the other terminal of the generator and, before delivering the fields:
each catheter with electrodes is pushed into the tissues in order to penetrate into the cavity; the needle of each catheter is then slid so as not to damage the walls of the cavity during the shifting of the catheter electrode inside the cavity and during the delivery of the fields;
the catheter electrodes being pushed in so as to be approximately parallel; and,

active ingredient is injected using at least one catheter electrode.

It is an embodiment of the methods of the present invention that at least one non-invasive electrode is also connected to the terminals of the generator and placed on the tissues bordering the cavity.

In another embodiment of the methods of the present invention, the non-invasive electrode is pressed against the tissues in order to get closer to each catheter electrode.

In a further embodiment of the methods of the present invention, at least one of the electrodes is a conductive catheter electrode used to inject the active ingredient having previously removed the needle from the catheter.

In yet a further embodiment of the methods of the present invention, at least one needle of a catheter-electrode is used to inject the active ingredient.

In a still further embodiment of the methods of the present invention, at least one of the electrodes is a conductive catheter electrode and in that the needle is fully withdrawn from its respective catheter before the delivery of the fields.

It is within the scope of the methods of the present invention that the active ingredient is injected continuously as the electrodes are pushed in.

It is additionally within the scope of the methods of the present invention that the active ingredient is injected in successive stages as the electrodes are pushed in.

In one embodiment of the methods of the present invention, at least one invasive electrode is gripped using a casing thus allowing it to be electrically connected to its terminal of the generator and allowing a good grip on the electrode.

In another embodiment of the methods of the present invention, once the catheter electrodes are introduced into the cavity, a means is used to modify the angle of the axis of the electrodes in order to obtain a good parallelism and prevent any risk of contact between electrodes connected to different terminals of the generator.

In yet another embodiment of the methods of the present invention, once the catheter electrodes are introduced into the cavity, a physical device is applied to the end of the electrodes not penetrating into the tissues in order to modify their respective angle and make them parallel.

In a further embodiment of the methods of the present invention is that a means is used to ascertain the distance between the ends of the electrodes, and the relative position of the electrodes is adapted in order to obtain the desired electric field or electric current according to the targeted tissues.

In a further still embodiment of the methods of the present invention, a means is used to ascertain the distance between the ends of the electrodes, and the programming of the generator is adapted in order to apply the voltage allowing the desired electric field or electric current to be obtained according to the targeted tissues.

It is also within the scope of the methods of the present invention that the generator has a means of continuously ascertaining the distance between the ends of the electrodes, and can dynamically modify the programming of the pulses in order to apply the parameters allowing the desired electric field or electric current to be obtained according to the targeted tissues.

It is further within the scope of the methods of the present invention that the invasive electrodes are parallel, made integral and held in place with the help of a casing allowing a good grip on the device and allowing the electrodes to be connected to their respective generator terminal.

In one embodiment of the methods of the present invention, the first group of electrodes is composed of an invasive electrode, and that at least one electrode of the second group of electrodes is non-invasive and is positioned on the surface of the tissues containing the ingredient.

In another embodiment of the methods of the present invention, at least one invasive electrode of the first group of electrodes is pushed through organs in order to reach the zone of tissues containing the active ingredient without passing through the non-invasive electrode.

In a further embodiment of the methods of the present invention, the invasive electrodes are pushed along an axis forming part of a plane substantially parallel to the plane occupied by the surface of the plurality of non-invasive electrodes in contact with the zone of tissues containing the active ingredient.

In yet a further embodiment of the methods of the present invention, the upper part of at least one invasive electrode is electrically insulated in order to prevent the passage of stray electric current into the volume of tissues situated on the one hand between the electrodes and situated on the other hand between the surface of the skin and the zone of tissues containing the active ingredient.

It is within the scope of the methods of the present invention that the upper part of all invasive electrodes is electrically insulated in order to prevent the passage of stray electric current into the volume of tissues situated on the one hand between the electrodes and situated on the other hand between the surface of the skin and the zone of tissues containing the active ingredient.

It is additionally within the scope of the methods of the present invention that there are non-invasive electrodes of the first group of electrodes and of the second set of electrodes on the surface of the tissues covering the zone containing the active ingredient, the electrodes being applied to a single face of the tissues, so as to permit the delivery of fields spreading under the surface of the tissues to which the electrodes are applied.

In one embodiment of the methods of the present invention, a set of non-invasive electrodes is pressed against the surface of the tissues containing the active ingredient in order to modify their geometry in order to increase the volume of tissues located between the electrodes.

In another embodiment of the methods of the generator is programmed to emit alternatively sequences of electric pulses between each close pair of electrodes:
in order to obtain, between each pulse emitted by the generator in the zone where the tissues containing the active ingredient are located, an interval of less than 50 ms;
while still having, between two pulses at a unitary electrode pair, an interval greater than 100 ms;
in order to make the electric pulses even less harmful.

In a further embodiment of the methods of the present invention, the duration between each electric pulse emitted by the generator to a pair of electrodes is comprised between 1 ms and 50 ms, in order to reduce the harmfulness and reduce the intensity of the muscular contractions.

In yet a further embodiment of the methods of the present invention, the electric pulses emitted by the generator are unipolar and have a square shape.

In an additional embodiment of the methods of the present invention:
the ratio between the potential difference between each electrode and their distance is comprised between 10 volt/cm and 750 volt/cm,

the duration of the electric pulses is comprised between 1 and 250 ms,

the duration between the electric pulses is comprised between 1 and 1500 ms,

the number of pulses of each sequence of pulses is comprised between 1 and 1000.

It is also within the scope of the methods of the present invention that the volt/cm ratio applied between the electrodes assumes a value comprised between 1.05 and 1.50 times the optimum value of the fields for the targeted tissues in order to increase the volume of tissues containing the active ingredient passed through by fields at an optimum number of volt/cm.

In another embodiment of the methods of the present invention, a tranquillizer is injected before triggering the pulses with the help of the generator. In one embodiment, the tranquillizer used is xylazine.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIGS. 1a and 1b show diagrammatically the way in which an invasive electrode is put in contact with the tissues.

FIGS. 1c and 1d show diagrammatically the way in which a non-invasive electrode is put in contact with the tissues.

FIGS. 1e and If show diagrammatically the electrotransfer method, with a device having 2 electrodes, seen respectively in profile and from above.

FIG. 1g shows diagrammatically the electrotransfer method, the active ingredient being located between the electrodes (seen from above).

FIGS. 2a and 2b show diagrammatically the zones covered by fields, the distance between the electrodes being double in FIG. 2.a, with a constant v/cm ratio.

FIGS. 3a, 3b and 3c show diagrammatically an example of a device where the 2 electrodes are respectively positioned at a distance from, bordering and inside the active ingredient.

FIGS. 4a and 4b show diagrammatically an example of a device with one electrode positioned in the centre of the active ingredient and having the opposite sign to the other electrodes bordering the active ingredient, showing respectively a profile view of a device with 3 electrodes, and a view from above of a device with 9 electrodes, the electrodes all being of the same depth.

FIGS. 5a and 5b illustrate the fields delivered for a device with one electrode placed between 2 (FIG. 5a) or 4 (FIG. 5b) electrodes of opposite sign.

FIGS. 6a, 6b, 6c and 6d illustrate the fields delivered with a volt/cm ratio respectively less than, equal to, greater and much greater than the optimum ratio for the type of tissues treated.

FIGS. 7 a and 7b illustrate the fields delivered for a device composed of two pairs of electrodes, the electrode pairs delivering a volt/cm ratio equal to (FIG. 7a) and 20% greater than (FIG. 7b) the optimum ratio for the type of tissues treated.

FIGS. 8a and 8b illustrate an example of a device composed of 3 electrodes and one (FIG. 8a) or two (FIG. 8b) injection needles positioned in the centre of the device at intermediate depths.

FIGS. 9a, 9b and 9c illustrate an example of a device integrating an electrode-injection needle in the centre, the active ingredient being respectively delivered once the electrodes are pushed into the tissues (FIG. 9a), or delivered to an intermediate depth (FIG. 9b), the electrodes being pushed in up to the guard (FIG. 9c).

FIGS. 10.1 and 10.2 illustrate an example of a device the 3 invasive electrodes of which (respectively the central electrode) are electrically insulated over one part using a plastic film.

FIG. 10.3 illustrates an example of a device of which the outside electrode 10 is non-invasive, the central needle electrode electrically insulated over a part using a plastic film.

FIG. 11 illustrates an example of a device with integral electrodes, provided to be used without a casing.

FIGS. 12a to 12c propose an example of pieces making up a device according to the invention including a casing and a stop system.

FIG. 13 illustrates an example of an injection piece placed in its housing of the casing.

FIG. 14 illustrates an example with a locking ring sliding along the closed casing.

FIG. 15 illustrates an example of a locked device ready to be applied to the subject.

FIG. 16a illustrates an example of a catheter electrode the needle of which is respectively in the catheter, slightly shifted so as not to damage the tissues or the walls, and withdrawn from the catheter.

FIG. 16b illustrates an example of a double-needled conductive catheter electrode, the active ingredient being delivered through the catheters.

FIGS. 17a to 17c illustrate examples of conductive catheter electrodes.

FIGS. 18a to 18c illustrate an example of use of the device with a conductive catheter electrode in a cavity.

FIG. 18d illustrates an example of means for making two catheter electrodes approximately parallel.

FIG. 18e illustrates an example of an electrode partly covered by a non-conductive catheter.

FIG. 19 shows diagrammatically an example of “rectangular” unipolar pulses.

FIGS. 20a and 20b illustrate the field lines spreading under the surface of the tissues, between non-invasive electrodes with electrodes in form of a wire or tip, over a flat and dished surface respectively.

FIG. 21 illustrates an example of a device composed of a non-invasive electrode and an invasive electrode, the latter passing through the tissues in order to reach the zone of tissues, along an axis approximately parallel to the plane of the non-invasive electrode placed on the tissues.

FIG. 22 illustrates an example of a device composed of a non-invasive electrode and an invasive electrode, the latter passing through the tissues in order to reach the zone of tissues, along an axis approximately perpendicular to the plane of the non-invasive electrode placed on the tissues.

FIG. 23 illustrates an example of a device composed of a non-invasive electrode and an invasive electrode, the latter passing through the non-invasive electrode through an orifice, and the position and the angle of the axis of the needle of which is guided by a guide device.

FIGS. 24a and 24b illustrate examples of non-invasive electrodes having respectively the form of a disk pierced by an orifice in its centre and of a horseshoe.

FIG. 24c illustrates an example of non-invasive electrodes composed of several non-integral flat electrodes.

FIG. 24d illustrates an example of a device composed of two parallel wire-like electrodes held in place in place by an adhesive strip.

FIG. 24e illustrates an example of a device composed of two wire-like electrodes held in place by a sheath.

FIG. 24f illustrates an example of a device composed of a group of non-invasive electrodes arranged in a chequerboard pattern and held in place by a sheath.

FIG. 25 illustrates an example of a device composed of non-invasive wire-like electrodes along a non-parallel axis.

FIG. 26 illustrates an example of a device composed of non-invasive electrodes made integral by forceps allowing the geometry of the tissues to be pressed and deformed.

FIG. 27 illustrates an example of a device composed of two integral non-invasive electrodes connected to the same terminal of the generator, being pressed against the tissues in order to get closer to a catheter electrode connected to the other terminal of the generator and located in a cavity.

### DETAILED DESCRIPTION

The present invention allows a solution of active ingredient to be injected into all tissues, in particular into muscles, tumors, joints, the dermis and the epidermis, and into all organs, with the exception of the heart, and in particular the bladder, the stomach, the kidneys, the lungs, all the organs of the head, including in particular the brain, the ears, the eyes, the throat, of a animal or human, living or not, and the delivery of fields in order to permeabilize the tissues and promote the transfer of the active ingredient into the cells.

The device with electrodes according to the invention is composed of:
a first group of electrodes 11 composed of at least one electrode, the electrodes being connected to one terminal of a generator of electric pulses 21
a second set of electrodes 10 composed of at least one electrode, the electrodes being connected to the other terminal of the generator of electric pulses 21, and therefore of a different voltage to the first group of electrodes.
a means of injecting the active ingredient 36.

The means of injecting the active ingredient can be composed of one or more needles. It can also be a means of introducing the active ingredient under pressure, using, for example, a compressed-air pistol, by patch or any other means known to a person skilled in the art.

In the present application, the expression “electrode” designates any type of electrode, in particular solid or hollow, invasive or non-invasive. The term “invasive electrode” designates an electrode designed to penetrate the inside the tissues. The term “non-invasive electrode” designates an electrode designed to remain on the surface of the tissues.

The two groups of electrodes 11, 10 are composed of invasive electrodes and/or non-invasive electrodes.

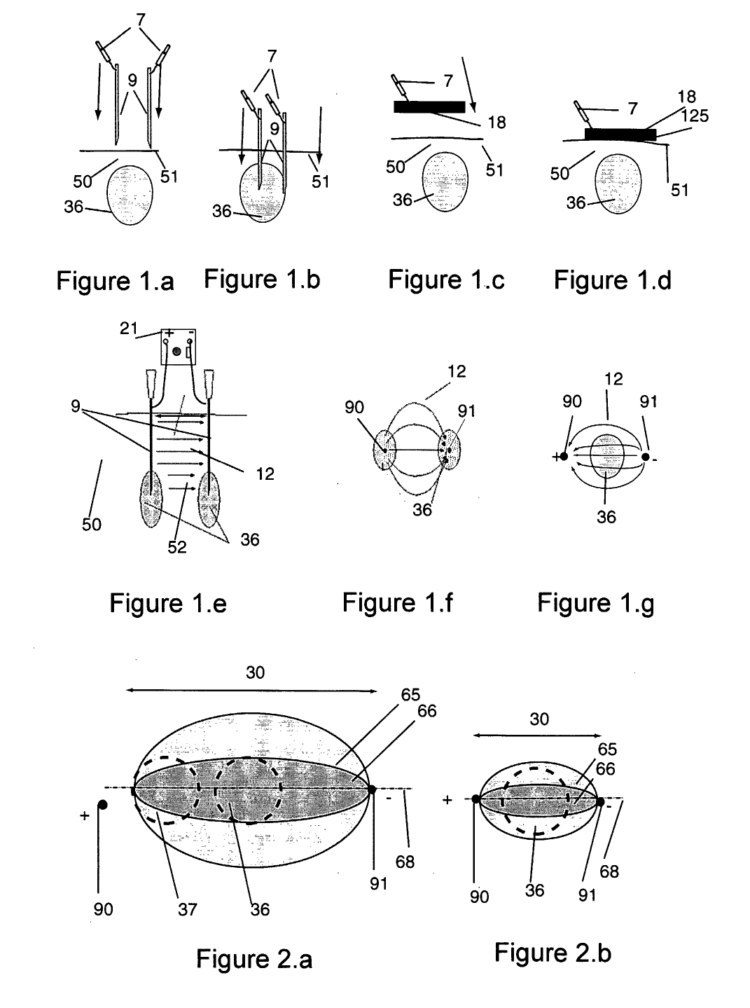

As shown by way of example in FIGS. 1a to 1d, in the present application, by “put an electrode in contact with the tissues to be treated”, is meant firstly: when the electrode is invasive (9), to push it into the tissues (50) of the subject. It can be pushed inside the zone of tissues that must contain the active ingredient, and can also be pushed in by the edge, even close by. Otherwise, when the electrode is non-invasive (18), what is meant is: to place the electrode on the surface of the tissues concerned (51). A layer of conductive gel can also be inserted (125) between the electrode and the tissues in order to improve conductivity. In both cases, the electrode is in electric contact with the tissues. In the case of a plurality of electrodes put in contact with the tissues to be treated, these can, depending on their form, be pushed into the tissues or placed on the surface of the tissues successively or simultaneously. To put an electrode in contact with the zone of the tissues to be treated implies that the active ingredient can be injected before, while or after the electrode is put in contact with the tissues. In the case of a plurality of electrodes, the active ingredient can be injected in its totality or in part before, while or after each electrode is put in contact with the tissues. The electrode is connected to the generator by an electric wire (7) or any other means known to a person skilled in the art.

The electric pulses delivered can have a square, unipolar, bipolar, exponential or other form. The generator generates for each pair of electrodes one or more predefined sequences of electric pulses, fewer than 100 in number, each pulse preferably being characterized by the following elements which can vary between two pulses, or remain identical within a single sequence:
volt/cm ratio (difference in voltage/distance between the two electrodes) comprised between 10 v/cm and 1 500 v/cm
the voltage difference between each pair of electrodes can be from 1 v o 2 500 v
duration of the pulse, comprised between 1 and 1000 ms
duration between two pulses, comprised between 1 and 2000 ms
distance separating the electrodes in the zone where the active ingredient is injected less than 10 cm

The period between two pulse sequences is less than 600 sec, and the number of sequences is less than 25. The intensity delivered will be less than 5 amperes.

The invasive and non-invasive electrodes are composed of metal materials, preferably stainless and of medical grade. They can be solid or hollow, for example medical injection needles.

An example of a device with 2 invasive electrodes 90, 91 is described in FIGS. 1.e and 1f.

For an optimum efficiency for the electrotransfer, the active ingredient must be passed through in its entirety by the fields 12, and therefore be located between the electrodes, as represented in FIG. 1.g for a device with 2 invasive electrodes 90, 91. As represented in FIG. 2.a, the central zone 36 is better covered by the fields than an eccentric zone 37, for obvious reasons of geometry. The active ingredient must preferably be located in the centre of the device with electrodes.

The efficiency of the electrotransfer depends on the value of the fields, defined by the ratio between the voltage between electrodes and the distance 30 between the 2 poles (electrodes), and of which the unit is: volt/cm. This ratio must approach an optimum value dependant on the nature of the tissues, and more particularly on the size of the cells making up the tissues. In the present application this value is called “direct optimum field”. The tissues 66 covered by the fields according to this ratio are represented in dark grey in the diagrams. With a smaller ratio, efficiency tends to reduce progressively, as the force of the fields becomes too small. With a greater ratio, the gains are counterbalanced by the toxicity of the hyperpermeabilization of the cells leading to cell death, and efficiency then tends to reduce. The tissues 65 covered by the fields according to this less optimum ratio (thus due to a volt/cm ratio that is too great or too small) are represented in light grey in the diagrams of the present application.

The fields delivered 12 between two electrodes occupy a volume having an approximately oval shape along the axis 68 connecting the two electrodes. The further the field lines are from this axis, the greater the distance covered, and therefore the more the volt/cm ratio decreases.

The distance between the electrodes must be minimal for reasons of harmlessness. In fact, a large distance means:
A proportional increase in the voltage between the electrodes (90, 91) which can then reach dangerous limits.
A diffusion of the current over a larger surface. This is illustrated by FIG. 2.a, where the voltage and the distance between the electrodes is doubled compared with FIG. 2.b. The current passes through more innervated surface, resulting in more contraction, and engendering a more intense painful reaction.

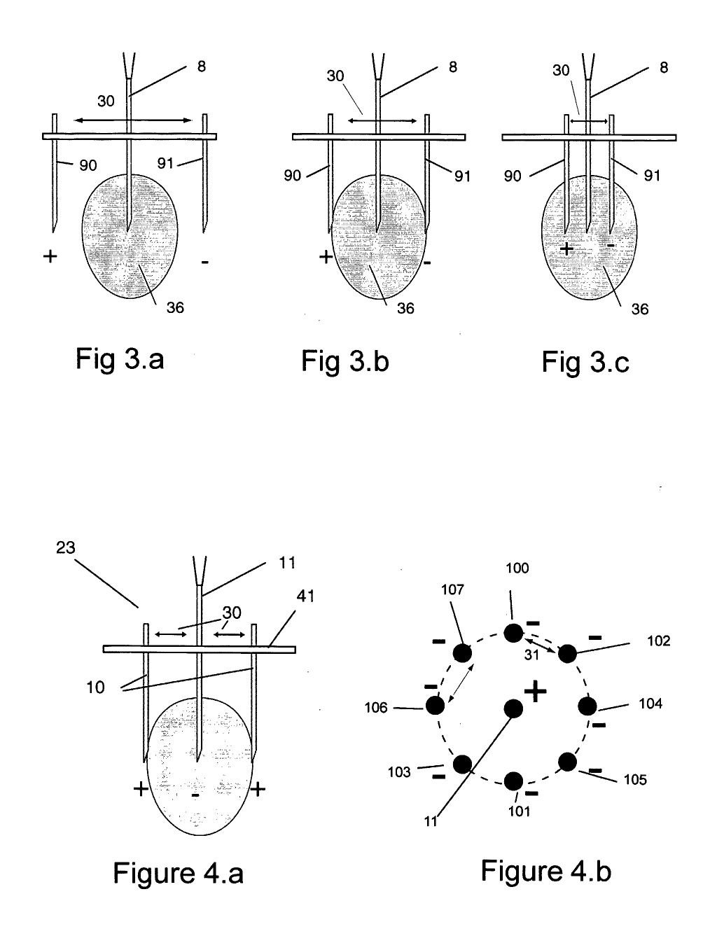

In order to have a minimum distance 30 between the electrodes, while still maintaining efficiency (maximum of active ingredient passed through by the fields), the electrodes must border the injected active ingredient 36. This is illustrated by FIGS. 3.a to 3.c, showing by way of example a device in which the injection needle 8 of the active ingredient is to be found centred between the two invasive electrodes 90, 91.

The invention proposes a device where the first group of electrodes 11 is introduced within the active ingredient, preferably in its centre, and is surrounded by the second group of electrodes 10. An example is illustrated in FIG. 4.a with invasive electrodes. The distance 30 between the electrodes is halved compared with the previous device (FIG. 3.b). If the electrodes of the second group are non-invasive, then the second group of electrodes is positioned on the surface of the tissues, on the site of injection of the active ingredient.

In a preferred form of the invention, if the device is constituted by more than one invasive electrode, then the invasive electrodes are all the same depth.

In order to improve the efficiency of the transfer of active ingredient into the cells, the electrodes of the second group 100 to 107 must be distributed evenly, forming a circle of which the central electrodes 11 form the centre. They must therefore be located at a constant distance 31 from each other, as illustrated in FIG. 4.b.

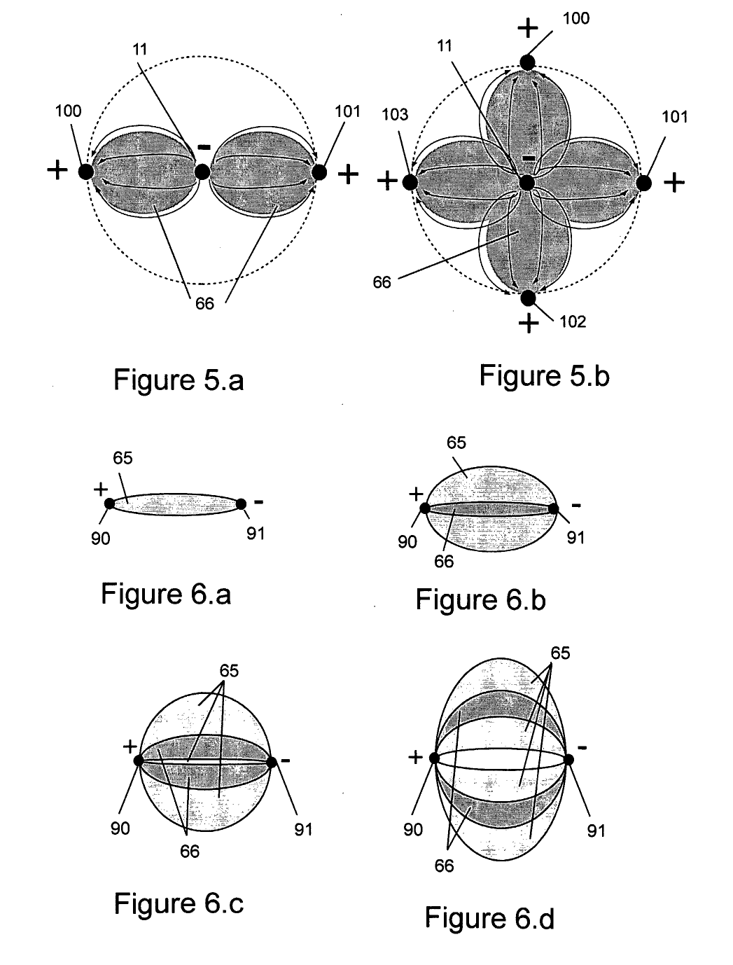

FIGS. 5.a and 5.b are preferred forms of the invention, for invasive or non-invasive electrodes, and illustrate the surface electrotransferred with a device where the second group of electrodes is composed respectively of 2 electrodes (100, 101) and 4 electrodes (100 to 103). The fields delivered by the latter device cover a greater surface, close to a sphere.

The location of the zones treated with an optimum efficiency 66, and with an average efficiency 65, depends on the volt/cm ratio applied to the pair of electrodes:
In FIG. 6.a, the volt/cm ratio between the electrodes is smaller than the direct optimum field.
In FIG. 6.b, the volt/cm ratio between the electrodes is equal to the direct optimum field.
In FIG. 6.c the volt/cm ratio is slightly greater than the direct optimum field, and therefore a larger zone located between the electrodes is treated with optimum efficiency 66, the adjacent zones with average efficiency 65.
In FIG. 6.d the fields are greater than the direct optimum field, and their significant toxicity wipes out the effects of the electrotransfer on the tissues placed on the axis between the two electrodes.

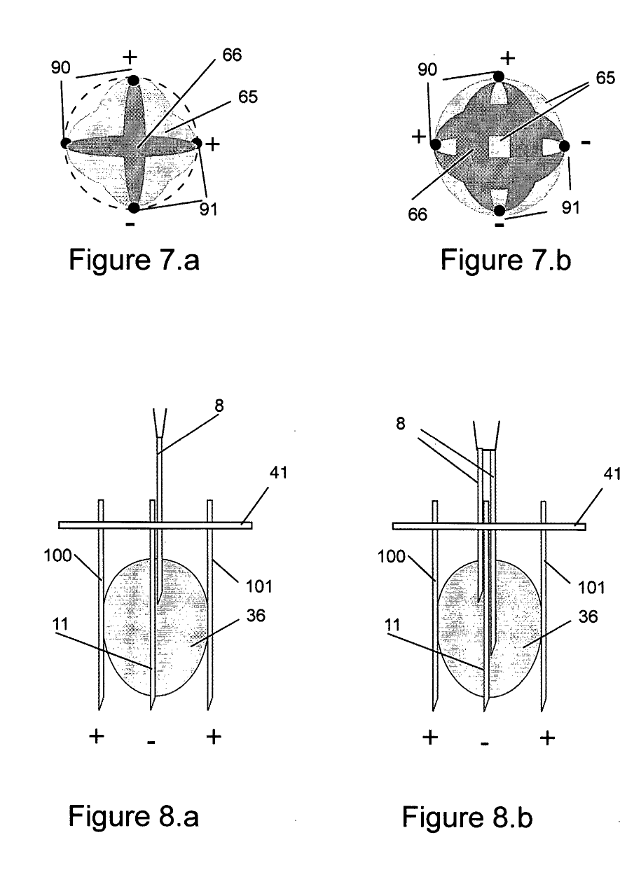

It is thus seen that the best efficiency is obtained if the volt/cm ratio applied between the electrodes is slightly greater (also shown by way of example in FIG. 7.b, for a ratio comprised between 105% and 150%) than the direct optimum field compared with the results obtained by applying the direct optimum field between the electrodes (example FIG. 7.a). However, this method can be applied only if the growth of the electric field in volt/cm remains tolerable for the subject in terms of harmlessness and toxicity.

In a device with several pairs of invasive electrodes located in the same zone (shown by way of example in FIGS. 7.a and 7.b for a device with 2 pairs), the pulse sequences can be delivered successively, simultaneously or overlapping. The

In the case of a device where the second group of electrodes is composed of at least 2 pairs of electrodes, efficiency is also improved if the pairs of electrodes successively transmit the fields, compared with a simultaneous delivery of the fields, certain tissues being treated twice.

In order to precisely position the first group of electrodes in the centre of the active ingredient, the injection needle 8 of the active ingredient as well as the electrodes 11, 100, 101 can be kept in their respective places with the help of one or more clamping pieces 41 which allow them to be kept in a defined geometry. This is shown by way of example in FIG. 8.a for a device composed of invasive electrodes.

In the remainder of the description, unless otherwise stated, the electrodes of the first group of electrodes 11 are invasive. The examples will illustrate whole devices or those that are part of a device containing more electrodes. The invasive needle-electrodes of the first group of electrodes will be represented in the diagrams by a single needle-electrode 11. The first set of electrodes, located in the centre of the device, and including the means of injecting the active ingredient, will sometimes be called “first electrodes”, the second group of invasive electrodes will sometimes be called: “outer electrodes”.

In order that the active ingredient is well situated between the electrodes, as shown by way of example in FIG. 8.a on a device with 3 invasive electrodes, the injection needle 8 must be at a depth less than that of the central electrodes and/or of the outer electrodes. FIG. 8.b shows by way of example a device with several injection needles 8, positioned against the central needle electrode 11, and arranged regularly at depths less than those of the electrodes in order to distribute the active ingredient evenly between the electrodes. It can be seen that the injection needles, being in contact with the central electrode, also serve as an actual electrode. In this example, the electrodes and needles are made integral with the help of a support (41).

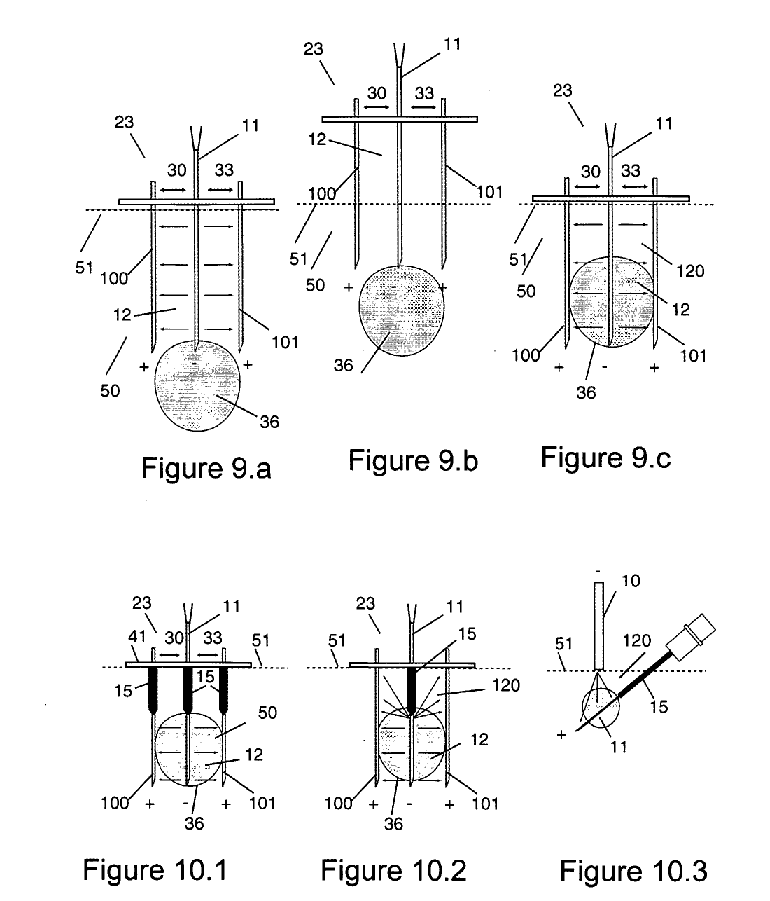

The central electrode can also serve as an injection needle. However, the drawback of this device is that some of the active ingredient is diffused below the central needle electrode, and is no longer passed through by the fields (FIG. 9.a). A method proposed by the invention is to inject the active ingredient 36 at one or more intermediate depths in the tissues (50). The device with electrodes 23 is pushed more deeply into the tissues at each stage, in order to inject the active ingredient. At the last stage, the electrodes are pushed in up to the guard without delivering active ingredient. This method thus allows the active ingredient to be injected into a zone which will be optimally positioned between the electrodes. This method is shown by way of example in FIGS. 9.b and 9.c for a device with invasive electrodes (100,101, 11), and with a single intermediate stage where all the active ingredient is injected.

Through the deep injection of the active ingredient, in particular into the muscles of a large mammal or human, fields 120 are also diffused in tissues that do not harbour any active ingredient. This is illustrated in FIG. 9.c. But this results in a toxicity and a contraction or pain which is unhelpful and unbeneficial for the patient. The invention proposes to apply an electric insulating material 15 to the upper part of the needles 100, 101, 11, as shown by way of example in FIG. 10.1 for a device with 3 invasive electrodes, which greatly limits the quantity of current passing through the tissues. This insulating material also has the beneficial effect of insulating the electrodes vis-a-vis the surface of the skin, in order to prevent the fields from spreading just under the skin 51 of the subject. The fact of insulating only the needles connected to a single terminal of the generator will only reduce some of the stray currents 120, as illustrated in FIG. 10.2 for an example of a device with 3 invasive electrodes. It may also be of advantage to insulate the upper part of the central electrode needle for a device comprising one or more non-invasive electrodes (an example of a device is illustrated in FIG. 10.3).

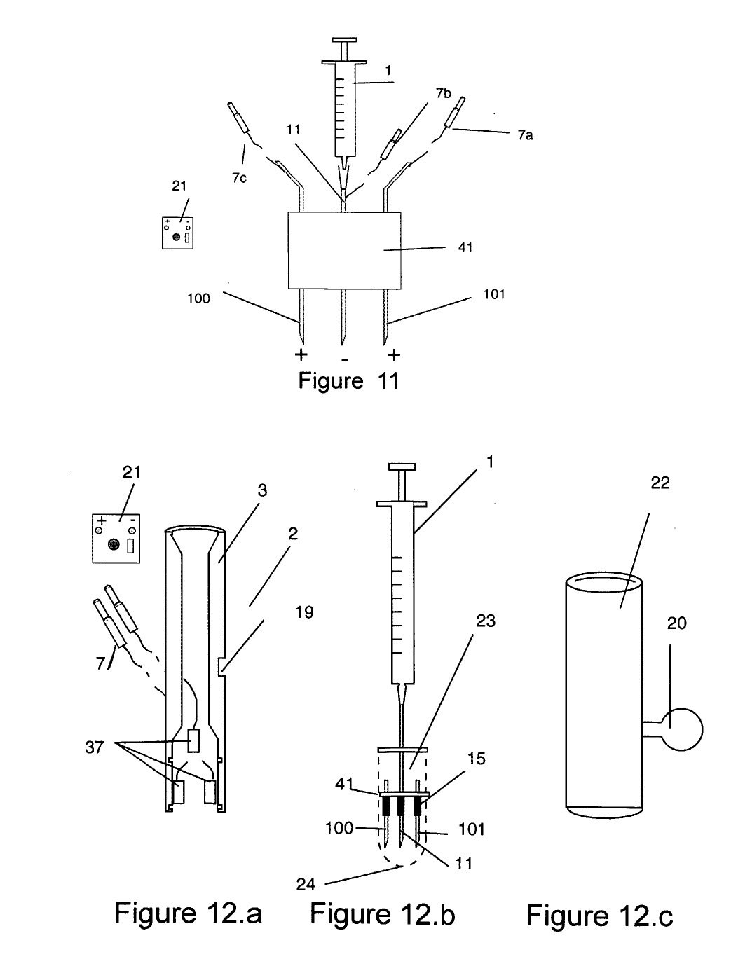

The needles and the invasive or non-invasive electrodes can be made integral and clamped by a rigid or slightly flexible support (41). The invasive components will then be parallel. An example is illustrated in FIG. 11 for a device with invasive needles (100, 101), the electrodes and needle-electrodes passing through an electrically insulating block 41, while still being directly connected to the generator 21 via electric wire 7a, 7b.

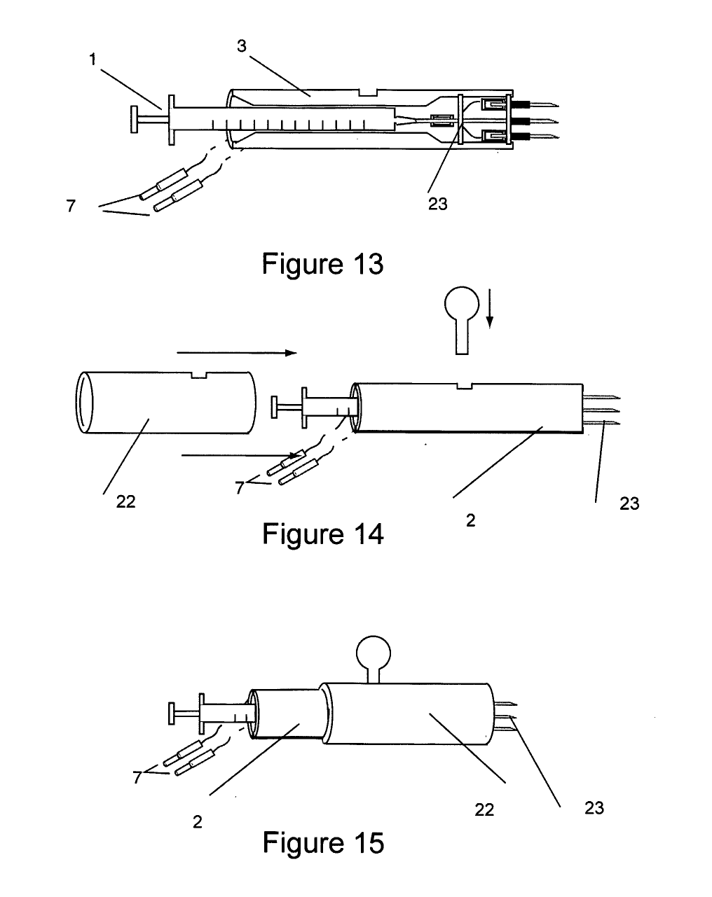

With a view to disposable and economical electrodes, the device can also comprise a casing 3 allowing the electrodes to be connected to the generator. This casing will allow an easily manageable grip on the device with electrodes and insulate the operator from electric currents. The casing will also be able to integrate the means of injecting the active ingredient and its reservoir 1. Moreover, it will be able to allow the device with electrodes to be pushed in to predefined depths, for example using stops. At each stage, except the last, it will allow the injection of a portion of the active ingredient which will be homogeneously distributed between the electrodes (or between the non-insulated part of the electrodes). An example of a device with disposable electrodes 23, with a casing 3 with its system of stops 20-22 is presented in FIGS. 12.a, 12.b and 12.c. The method of assembly of this example of casing and electrodes and its stop is presented in FIGS. 13 to 15.

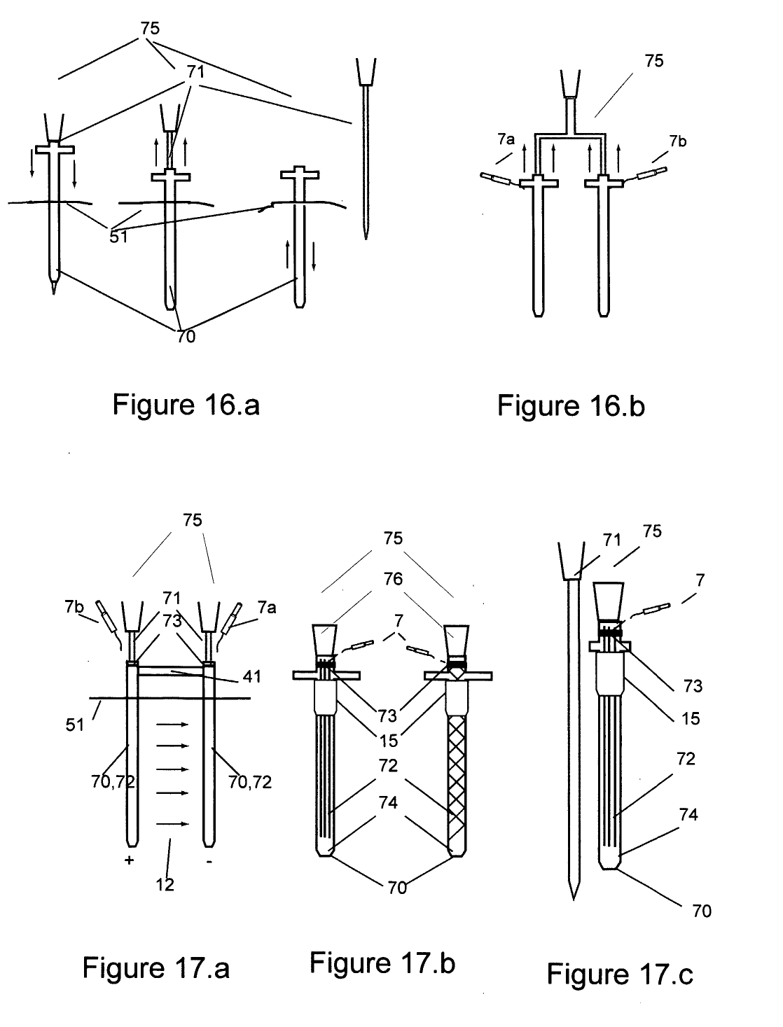

The invasive electrodes must be suitable for the treatment of fragile zones so as not to damage the treated zone with the needle during the muscular contraction caused by the delivery of the fields (comprising the tissues passed through by the electrodes and the walls 77 bordering these tissues). In fact, some tissues and walls reconstitute themselves little or not at all, and therefore steps must be taken to avoid damaging them during the operations for the positioning of the electrodes and the injection of the active ingredient and delivery of the fields. The device of the invention, called “catheter electrode”, an example of which is illustrated in FIGS. 16.a, 16.b, 17.a to 17.c, makes it possible to avoid this risk. FIG. 17.c describes all the components of a catheter electrode 75. The device is suitable for all tissues, in particular muscles, and is particularly indicated for organs composed of a sealed cavity 78 containing fluids, even viscous ones. This device is particularly indicated for the treatment of the synovial cavity of any joint of the limbs of all mammals of more than 2 kg, including humans.

A catheter electrode is an invasive electrode, composed of an invasive needle 71 covered by a catheter 70. A catheter is a tube composed of thin flexible materials having a greater or lesser resistance to compressive forces along its axis, for example silicone. It is provided to be introduced into the tissues with the help of the needle 71 placed inside the catheter. The surface of the catheter is made electrically conductive, while still preserving a degree of its flexibility. Once the catheter electrode is in the tissues, the needle is partly drawn back, even fully withdrawn from the device. The catheter 70 can then no longer damage the tissues. If the tissues are fluid, it can be pushed in and in some cases its direction adjusted.

In a variant of the invention, the active ingredient can be introduced simultaneously into several electrodes comprising the device, as shown by way of example in FIG. 16.b by an example of double-needled conductive catheter electrodes, the active ingredient being delivered through the catheters, the two electrodes being integral.

In a variant of the invention, the catheter electrodes can be composed of several needles and catheters. The catheters can be aligned, be arranged in the form of circle or be arranged in any other way. An example is illustrated in FIG. 17.a, where the two catheters are each connected to a different terminal of the generator, the support 41 gathering together the electrodes and not being electrically conductive, the needles being independent.

In order to be conductive, the catheter must be covered with film or metal wires or any other electrically conductive materials 72. The wire can be arranged in any form (braided, parallel, meshed etc.) envisaged by a person skilled in the art.

If the device is constituted by more than one catheter electrode, the electrodes can then be connected to a single support or be independent.

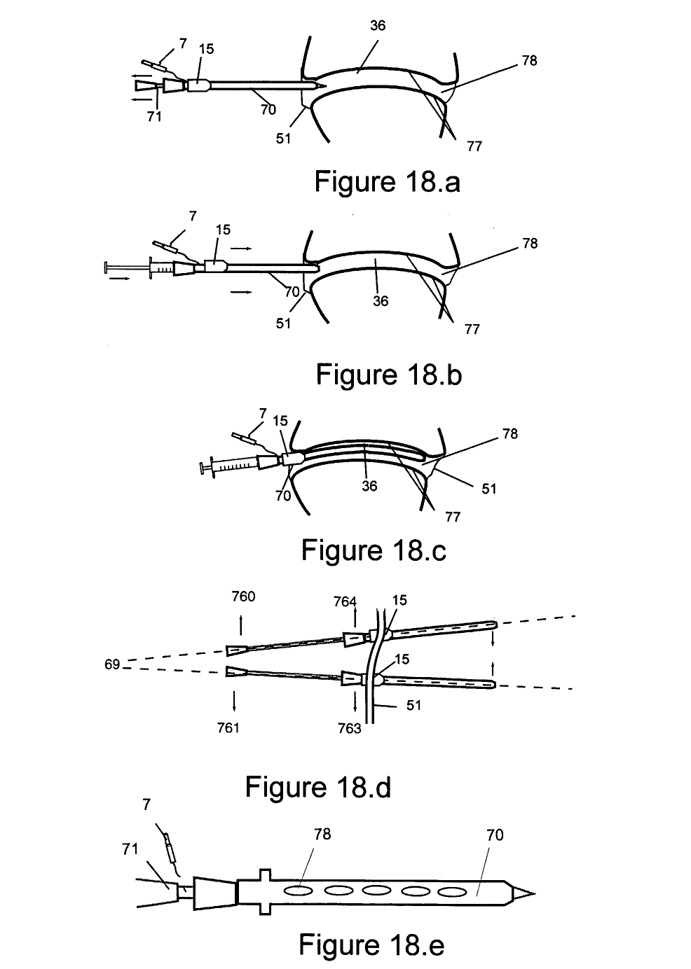

In the method shown by way of example in FIGS. 18.a to 18.d, the device can be slightly introduced into the cavity thanks to the invasive needles 71 which are then withdrawn. The active ingredient is then injected into the cavity through the inside of the catheter 70. It can be injected progressively as the electrode is pushed into the cavity. In the case of a synovial cavity the catheter, being flexible, allows the joint to be bent in order to satisfactorily distribute the active ingredient.

The active ingredient can be delivered once or several times, or continuously, as the catheter is pushed into the tissues.

In a variant of the invention, the needle 71 of each catheter 70 is slightly shifted, so as to no longer be able to re-establish contact with the walls of the cavity. However, the needle remains in the catheter, so as to make it more rigid while it is pushed into the cavity. In this case, the active ingredient can be injected progressively by the needle 71. Once the assembly is in position, the needles 71 can be withdrawn prior to the delivery of the fields.

When the generator delivers the electric pulses, the current passes through the metal surface of the catheters.

The catheters can be covered, in their upper part, by an electric insulating material 15 (shown by way of example in FIGS. 17.b to 18.d), in order that the fields spread only in the cavity. Under these conditions, if the walls of the cavity are not innervated, the subject feels practically no pain and experiences only a slight muscular contraction, and significant voltages can be delivered.

The catheter electrodes can be used on all types of tissues. On non-fluid tissues, and in particular muscles, the needle need not be removed before the device is pushed in up to the desired final depth.

If the device is composed of at least 2 catheters connected to different terminals of the generator, then it is preferable that the axes of the catheters are parallel. The catheters can be made integral with the help of a support or by using a casing. However, the use of integral catheter electrodes can be difficult for tissues that are difficult to reach. It is possible, once the first electrode is in place, to use a guide that forces the parallelism of the second electrode: the guide is applied to the emergent part of the needle which has been pushed in, in order to guide the following electrode through another orifice of the guide. This principle can be applied to any type of invasive needles.

Many methods and devices allow the distance between the ends (74) of the electrodes to be precisely ascertained. This can be displayed by medical imagery systems. Another technique is to insert non-invasive needle electrodes inside the catheters, and using them as an electrode in order to emit a slight pulse through their tip which passes beyond the catheter. This allows the resistance between the electrodes, and thus the distance, to be ascertained.

It is also possible to use a device in order to adjust the parallelism. For example, a device can be used that is composed of rigid rods 760, 761 that are introduced into the catheter in order to orient the catheter as shown by way of example in example in FIG. 18.d, or a suitable device pressing against the outer ends (76) of the electrodes 763, 764. A casing can also be used, which can also provide the electric contact between the catheter electrodes and the generator.

The generator will be able to control the intensity level emitted during the delivery of the fields, and thereby indicate the degrees of success of the operation, compared with pre-recorded charts or with the result of the first pulse emitted.

The device can comprise, supplementing the catheter electrodes, one or more non-invasive electrodes placed on the surface of the tissues bordering the cavity, connected to one of the terminals of the generator, in the knowledge that at least one catheter electrode is connected to the other terminal. This electrode can be non-invasive (for example a disk placed on the knee). The device can comprise, supplementing the catheter electrodes, an invasive electrode (for example a needle allowing the periphery of the hip to be reached) but only approaching the zone of tissues containing the active ingredient, without penetrating it In this latter case, this needle will be able to be covered by an electric insulating material at its end. If the device comprises several pairs of electrodes, the pulses between each pair can be delivered simultaneously or successively.

The catheter electrodes can also be pierced by orifices (78) in the wall of the catheter (70), this no longer requiring conductive cover. An example is illustrated in FIG. 18.e. In this case, it is the needle (71) that ensures the electric contact with the tissues to be treated, by being in direct contact with the tissues through the orifices. With this type of electrode, it is also recommended, for reasons of harmlessness, to slightly shift the needle inside the catheter before triggering the fields. In the present description, this type of electrode is called: electrode partly covered by a non-conductive catheter.

In the present description, by the term catheter electrode is meant all types of catheter electrodes, in particular electrodes partly covered by a non-conductive catheter and conductive catheter electrodes.

The remainder of the description takes account of all types of electrodes and electrodes assembly.

The use of the generator to emit low-voltage and short-duration test pulses before the delivery of the therapeutic pulses can be generalized to include all types of electrodes. These pulses allow the distance between the electrodes to be controlled by analyzing the generated current intensity, and taking account of the nature of the tissues and the geometry of the electrodes. This also allows the pulse sequences programme to be recalculated. The programme of sequences can also be recalculated in real time by data methoding means during delivery of the fields, in order to correct the remainder of the sequence. This can be useful, for example if the space between the electrodes varies because of muscular contraction.

In order to reduce the harmfulness of the device, the use of tranquillizers such as xylazine (for example xylazine 7 mg/ml) very greatly reduces the contraction of the muscles and the pain reaction caused by the delivery of the fields, and corresponds to an alternative means in particular when anaesthetics constitute a contra-indication.

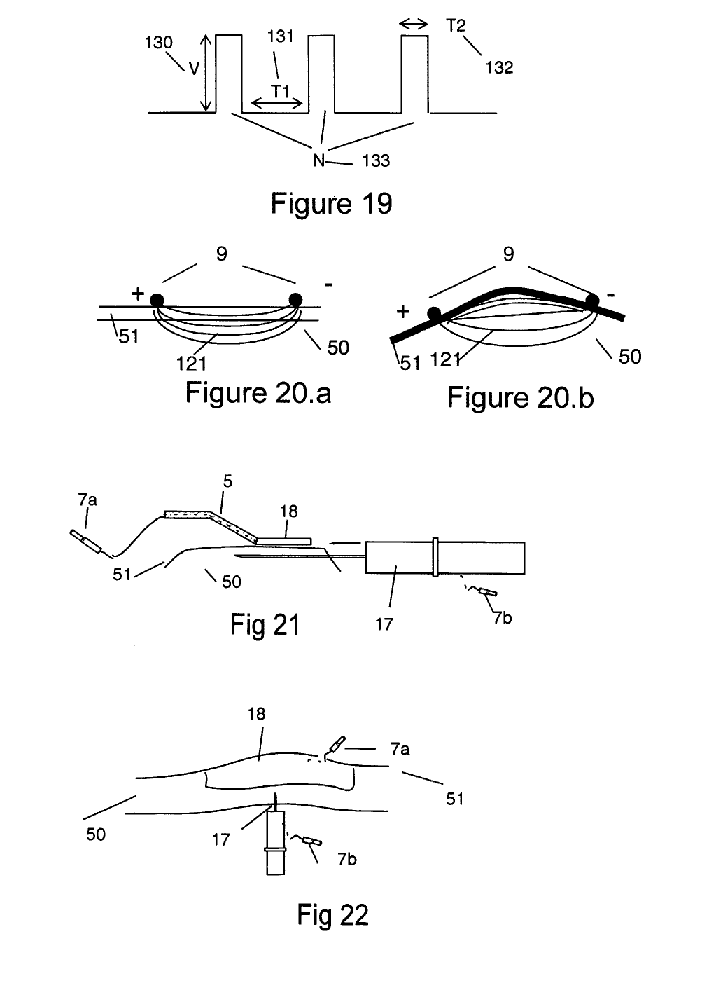

The sequences of pulses are defined by the following characteristics and parameters:
shape of the pulses: rectangular—unipolar shown by way of example by FIG. 19, sinusoidal, bipolar, exponential, etc.
the difference in voltage 130 between the electrodes, the number of pulses 133, duration of each pulse 132
time interval between each pulse 131.

The inventor found that the use of a short time interval between two electric pulses (comprised between 1 and 100 ms) very strongly reduced the contraction of the muscles and the pain reaction caused by the delivery of the fields, compared with the values customarily used (greater than 100 ms). This reduction in the muscular contraction and the apparent pain reaction is improved progressively as the interval decreases.

Experiments carried out on dogs and cats showed the following points:
The use of a tranquillizer leads to a very clear reduction in pain and muscular contractions,
The use of a time interval comprised between 1 and 50 ms leads to a very clear reduction in pain and muscular contractions, this reduction becoming greater as the duration decreases.

However, very short intervals can bring about a reduction in the efficiency of the electrotransfer. It is then possible to use a device with several pairs of electrodes, close, parallel or interlaced, the generator alternating the triggering of each pulse. In the present application, the pulses will be called “overlapped pulses”.

This allows, at the overall level of the zone of tissues, the simulation of a division of the duration between two pulses by the number of pairs of electrodes employed. It is recommended to stagger the pulses by a duration equal to the duration of the interval between the pulses divided by the number of pairs. A single electrode can be associated with several electrodes connected to another terminal of the generator to create several pairs of electrodes. For example, in the case of a device with 3 electrodes (ex FIG. 4.a, 5.b), each pair corresponds to the couple: central electrode, one of the outer electrodes.

In a preferred mode of the invention, all or some of the electrodes of the second group of electrodes are non-invasive, and arranged on the surface of the tissues covering (or close to) the zone containing the active ingredient containing the active ingredient.

The non-invasive electrodes can be integral with the invasive electrodes, or independent The two groups of electrodes can be constituted by invasive and/or non-invasive electrodes, the non-invasive electrodes being arranged on the surface of the tissues covering the zone containing the active ingredient.

The two groups of electrodes can also be constituted by non-invasive electrodes only.

In this case, in a preferred mode of the invention, the electrodes are applied to a single face of the tissues 51 containing the active ingredient so as to allow the delivery of fields 12 spreading under the surface of the tissues to which the electrodes 9 are applied. The fields delivered spread between the electrodes through a layer of intermediate tissues (skin, fat etc.) and pass through the tissues containing the active ingredient as exemplified by way of example in FIGS. 20.a and 20.b.

The non-invasive electrodes can be integral or independent; they can be used once only, or reused.

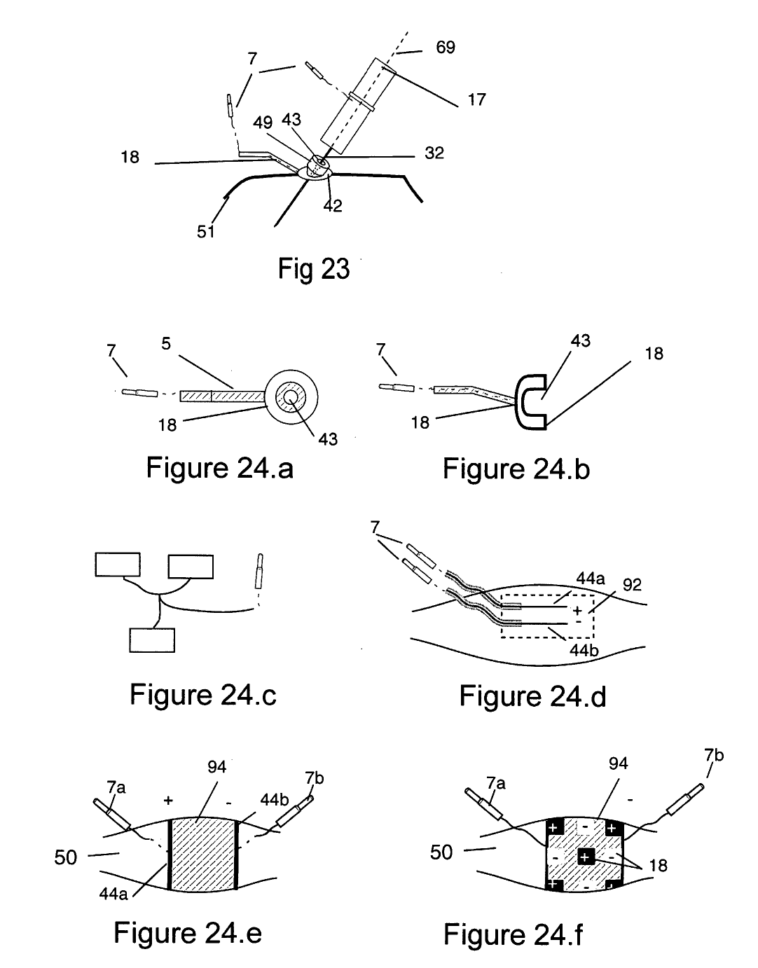

The invention proposes a device which comprises one or more invasive electrodes connected to the first terminal of the generator and one or more non-invasive needles connected to the second terminal of the generator, these electrodes not being integral. The invasive electrodes can pass through the tissues 50 in order to reach the zone containing the active ingredient. An example is illustrated in FIG. 21 where the axis of the invasive electrode 17 is substantially parallel to the plane followed by the surface of the non-invasive electrode 78 or by the surface of the tissues 51. An example is illustrated in FIG. 22 where the axis of the invasive electrode 17 is approximately perpendicular 18 to the plane followed by the surface of the non-invasive electrode or by the surface of the tissues. In a preferred form of the invention, a guide 49 can allow precise positioning of the place where the invasive electrode must penetrate the tissues and/or the angle of penetration of the axis 69 of the invasive electrode (shown by way of example in FIG. 23).

The surface of each non-invasive electrode 18 in contact with the tissues at the moment of delivery of the fields can be of any form. It can represent any geometry: rectangle, triangle, circle (shown by way of example in FIG. 24.a), semi-circle, arc of a circle, disk, horseshoe (shown by way of example in FIG. 24.b), oval, wire-like (shown by way of example in FIG. 24.d), symmetrical or asymmetrical etc. Each electrode can have an orifice 43 (an example of which is illustrated in FIGS. 24.a and 24.b), allowing the invasive electrodes to pass through it. FIG. 24.c illustrates a non-invasive electrodes example composed of several non-integral flat electrodes.

The non-invasive electrodes can supplement a device comprising invasive electrodes and catheter electrodes.

The surface of each non-invasive electrode in contact with the tissues at the moment of delivery of the fields can be of any size. It can be very small, such as a tip slightly flattened at its end. It can be larger, allowing for example the significant covering of the surface of the tissues containing the active ingredient, or the covering of a larger surface.

Each non-invasive electrode can be rigid or flexible. Each invasive electrode can be flat or slightly curved in order to follow the shape of the surface of the targeted tissues.

Each non-invasive electrode can be held against the tissues with the help of a sleeve 5, a casing, an elastic sheath 94 (an example of which is illustrated in FIGS. 24.e and 24.f), a bandage, an adhesive strip 92 (an example of which is illustrated in FIG. 24.d). FIG. 24.e shows an example of a wire-like electrode forming a circle. These accessories also serve to connect the electrode to the means 7, 7a and 7b of connecting it to the corresponding terminal of the generator.

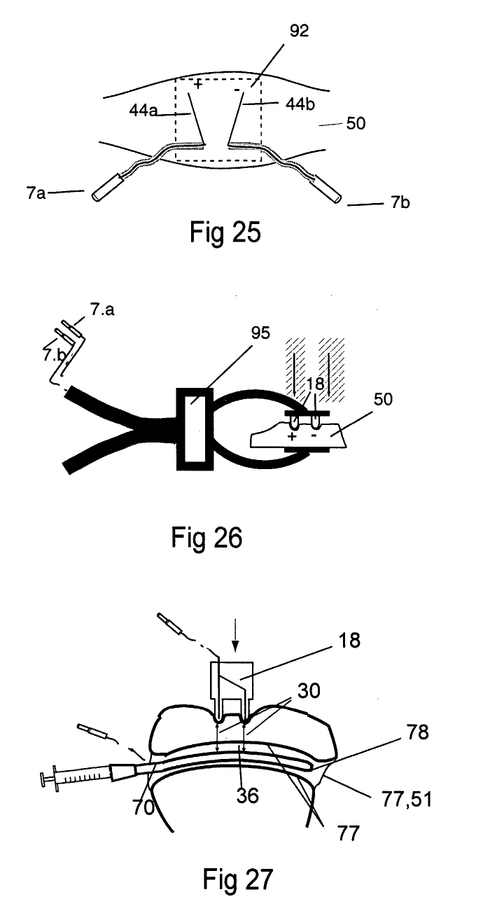

In a preferred example of the invention, the non-invasive electrodes can represent a symmetrical or non-symmetrical, even irregular, device. In the case where the electrodes are in the form of wires 44a, 44b and non-invasive, these electrodes can follow non-parallel axes (an example of which is illustrated in FIG. 25).

The non-invasive electrodes can be pressed against the tissues 50 in order to modify their geometry in order to increase the volume of tissues located between the electrodes, as is shown by way of example in FIG. 26 with an electrode support in the form of forceps.

The non-invasive electrodes can also be pressed against the tissues 50 in order to reduce the distance between these electrodes and other electrodes connected to another terminal of a generator, as shown by way of example in FIG. 27 with a double electrode. This makes it possible in particular to get close to an invasive needle introduced within a cavity.

One example of the invention is described in FIGS. 12.a, 12.b and 12.c. The device is composed of 2 main pieces:
The “injection piece” 23 bringing together in particular the electrodes, injection means and support
the housing 2 for the injection piece, called the “casing”.

The casing is composed of two half-cylinders 3, and the whole is fixed using a ring 22. The injection piece 23 is constituted by 3 aligned electrodes, parallel and at the same depth, the central electrode 11 being located at an equal distance between the two outer electrodes 100, 101 and allowing the active ingredient to be injected. An orifice is provided for receiving the syringe which will contain the active ingredient which will be injected through the first electrode (11)

The needles are held together using one or two clamping disks 41. An electric insulating film 15, comprising a thin wall made of synthetic material, is applied to the electrodes under the clamping disk 41.

The casing serves as a housing for the injection piece 23. It also serves as an electric connection with the electric terminals of the injection piece 2 and the electric wires 7 connected to the electric generator 21 for the electrotransfer. The two electrodes 100, 101 located on the periphery of the injection piece are connected to the same terminal of the generator, the first electrode to the other terminal. The casing is composed of one or more pieces, allowing, once assembled, an easy grip on the device to be achieved. The contact is produced by metal forms 38 pressed against the electrode by a spring or an elastic piece, or also by flexible metal plates. The casing also serves as a housing for the syringe 1 containing the active ingredient 36 to be injected between the two outer electrodes of the injection piece 23. A stop system 20 allows the first element to be pushed in to a predefined depth. FIG. 13 shows the device with the injection piece, composed of the syringe and the assembled injection piece, positioned in its housing.

The pulse sequence programme is defined, or a pre-recorded programme is selected which defines in particular the number and the duration of each pulse, the interval between the pulses, the synchronization of the sequences and the voltage to be applied between the electrodes.

The syringe and its active product are assembled with the electrode piece, and they are placed in the corresponding housing of the casing, then the two half-cylinders are assembled, then the casing is locked by slipping on the locking cylinder 22, FIG. 14, then the stop 20 is inserted (FIG. 15). The electric terminals 7 are connected to the generator of electric pulses 21, and the cap protecting the needles 5 is removed. The device is assembled (FIG. 15), and can then be implanted in the tissues of the subject, employing the medical practices and movements that are customarily used in order to implant a needle fitted with a syringe. The locking cylinder locks the device at a predefined intermediate depth. The active ingredient is injected, the stop is removed, and the casing is pushed in up to the guard.

The pre-programmed pulses series can now be started.

Then the casing is unlocked and the disposable items: syringe 1, injection piece 23 and its protective cap 5 are discarded.

There are many industrial applications for the administration of nucleotides aided by fields. The device according to the invention is to be used in particular for the administration of medicaments, in particular medicaments based on DNA, in human and veterinary medicine

The therapeutic applications in humans or animals concern, in particular, but quite clearly not exclusively, the treatment of tumors and the production of blood proteins.

The production of proteins in the blood is relevant to the treatment of hemophilia, growth disorders, myopathy, lysosomal and metabolic diseases in general, chronic renal insufficiency and beta-thalassemia by the endogenous production of erythropoietin. Other fields of application concern neoangiogenesis, atherosclerosis, using the protective effect of cytokines such as IT-10, vaccination, the use of antisense oligonucleotides, or also the prevention of peripheral neuropathy induced by cisplatin, by electrotransfer of a plasmid coding for a neurotrophin. Particular emphasis is currently placed on use in articular rheumatoid polyarthritis or in inflammatory pathologies in general, using the protective effect of IT-10, of anti-TNF or of other cytokines. The use of growth factors is also rich in potential in neurodegenerative or degenerative diseases of the joints (arthrosis).

Moreover, this is relevant to all pathologies that may benefit from the local expression of a secreted protein, such as for example an anti-inflammatory protein secreted in the joints for the treatment of arthritis, or an anti-angiogenic protein for the treatment of cancer. Similarly, production in a joint of growth factor can be envisaged for the treatment of arthrosis. The use of angiogenic proteins secreted locally for the treatment of peripheral arteritis can also be envisaged.

There is also relevance for the active and passive vaccination of humans and animals, the production of vaccines and the production of antibodies, in particular within the framework of pathologies connected with bioterrorism. Mention must also be made of therapeutic applications for which the intracellular expression of a protein is necessary, such as numerous neuromuscular diseases (myopathy), tumors, etc.

The invention will now be further described by the following numbered paragraphs:

1. A device for improving the in vivo penetration of molecules of active ingredient into the cells of the tissues of a human or animal subject, characterized in that it comprises:

a generator of electric pulses (21)
a first group of electrodes composed of at least one electrode (9) electrically connected to a first terminal of the generator of electric pulses,
a second group of electrodes composed of at least one electrode (9) electrically connected to the second terminal of the generator of electric pulses, and
a means (8) of injecting the active ingredient into the tissues.
   2. The device according to paragraph 1, characterized in that the first group of electrodes is composed of a single electrode (11).
   3. The device according to paragraph 2, characterized in that the electrode (11) is invasive.
   4. The device according to paragraph 3, characterized in that the electrode (11) has a means of injecting the active ingredient.
   5. The device according to paragraph 4, characterized in that the electrode (11) is an injection needle (8).
   6. The device according to paragraph 5, characterized in that a means of injecting the active ingredient is constituted by at least one integral needle (8) and placed close to the electrode and at a depth less than the depth of the electrode.
   7. The device according to paragraph 5, characterized in that:
the electrode is moreover constituted by a catheter envelope (70) pierced by orifices (78) of sufficient size to permit a contact between the needle and the tissues through these orifices;
the invasive needle of the electrode (71) passes through the catheter along its axis and can slide along the axis of the catheter and can be withdrawn from the catheter and,
the catheter and the needle, once assembled, form an invasive electrode, these electrodes being hereinafter called “electrode partly covered by a non-conductive catheter” (75).
   8. The device according to any one of paragraphs 4 to 6, characterized in that the electrode (11) is constituted by:
a catheter (70), covered by an electrically conductive surface (72) connected to the corresponding terminal of the pulse generator, hereinafter called “conductive catheter electrode”, its surface being composed of a material approximately retaining the flexibility of the catheter, which catheter can serve as means of injecting the active ingredient; and
an invasive needle (71) passing through the catheter along its axis and allowing penetration of the tissues and able to slide along the axis of the catheter and able to be withdrawn from the catheter, which needle can also serve as injection means.
   9. The device according to any one of paragraphs 3 to 8, characterized in that the invasive electrode is covered, in its upper part penetrating into the tissues, by an electric insulating material (15).
   10. The device according to any one of paragraphs 7 to 9, characterized in that at least one catheter electrode is magnetized (75).
   11. The device according to any one of paragraphs 3 to 10, characterized in that the second group of electrodes comprises at least one non-invasive electrode (18) arranged on the surface of the tissues covering the zone containing the active ingredient.
   12. The device according to paragraph 11, characterized in that at least one non-invasive electrode (18) has an orifice (43) allowing the invasive electrode (11) to pass through it.
   13. The device according to paragraph 12, characterized in that it also comprises a guide (49) allowing the axis of the invasive electrode (11) to be directed in a predefined direction.
   14. The device according to any one of paragraphs 3 to 10, characterized in that the second group of electrodes comprises a single invasive electrode (10).
   15. The device according to paragraph 14, characterized in that the electrode of the second group is an electrode partly covered by a non-conductive catheter (75).
   16. The device according to paragraph 14, characterized in that the electrode of the second group is constituted by a conductive catheter electrode (75).
   17. The device according to one of paragraphs 14 or 15, characterized in that the electrode of the first group is a catheter electrode (75) and, where the two needles are parallel, integral and connected by a support.
   18. The device according to any one of paragraphs 15 to 16, characterized in that the two invasive electrodes (10,11) are integral, assembled using a support (41), approximately parallel and approximately of the same depth.
   19. The device according to any one of paragraphs 4 to 10, characterized in that the second group of electrodes comprises a plurality of invasive electrodes (10).
   20. The device according to paragraph 19, characterized in that the invasive electrodes of the second group (10) are situated approximately on a circle of which the electrode (11) of the first group approximately forms the centre.
   21. The device according to one of paragraphs 19 or 20, characterized in that the invasive electrodes of the second group border the zone where the active ingredient is injected (36).
   22. The device according to any one of paragraphs 19 to 21, characterized in that two invasive electrodes (100, 101) of the second group of electrodes are substantially aligned with the electrode of the first group (11).
   23. The device according to any one of paragraphs 14 to 16, 19 to 22, characterized in that it also comprises a means (764, 763) allowing orientation of the electrodes along a parallel axis.
   24. The device according to any one of paragraphs 20 to 22, characterized in that all the electrodes (10, 11) are integral, assembled using a support (41), approximately parallel and approximately of the same depth.
   25. The device according to any one of paragraphs 14 to 24, characterized in that the device also comprises at least one non-invasive electrode connected to one of the terminals of the generator.
   26. The device according to any one of paragraphs 4 to 16, 19 to 23 characterized in that:
each invasive electrode can have a casing (2) allowing a good grip on the electrode and providing the electric connection between the electrodes and its terminal of the generator (21); and
the casing (2) having a housing allowing the electrodes and the means of injecting the active ingredient and the reservoir (1) containing the active ingredient to be accommodated.
   27. The device according to any one of paragraphs 4 to 16, 19 to 23, characterized in that:
a plurality of electrodes have a single casing (2) allowing a good grip on the electrode device and providing the electric connection between each electrode and its terminal of the respective generator (21);
the casing (2) has a housing allowing the electrodes and the means of injecting the active ingredient and the reservoir (1) containing the active ingredient to be accommodated.
   28. The device according to paragraph 23 or 27, characterized in that the casing also comprises the means of orienting the electrodes along a parallel axis.
   29. The device according to one of paragraphs 18 or 24, characterized in that:
the integral electrodes and their support have a casing (2) allowing a good grip on the electrode device and providing the electric connection between each electrode and its terminal of the generator (21); and
the casing (2) has a housing allowing the integral electrodes and their support and the means of injecting the active ingredient and the reservoir (1) containing the active ingredient to be accommodated.
   30. The device according to one of paragraphs 26 to 29, characterized in that the casing:
has a means allowing the electrode (11) to be successively pushed into the tissues to predefined intermediate depths;
and has a means allowing the injection of the active ingredient at each stop position.
   31. The device according to paragraph 30, characterized in that the means allowing the electrode (11) to be successively pushed into the tissues to predefined intermediate depths is constituted by at least one stop (20).
   32. The device according to any one of paragraphs 2 to 31, characterized in that a plurality of invasive electrodes connected to the generator are covered, in their upper part penetrating into the tissues, by an electric insulating material (15).
   33. The device according to any one of paragraphs 2 to 32, characterized in that all the invasive electrodes connected to the generator are covered, in their upper part penetrating into the tissues, by an electric insulating material (15).
   34. The device according to paragraph 1, characterized in that the two groups of electrodes comprise a set of non-invasive electrodes (18) arranged on the surface of the tissues covering the zone containing the active ingredient.
   35. The device according to the paragraph 34, characterized in that the electrodes are applied to a single face of the tissues (51) containing the active ingredient so as permit the delivery of fields spreading under the surface of the tissues to which the electrodes are applied.
   36. The device according to any one of paragraphs 1, 11 to 13, 25, 34, 35, characterized in that the surface in contact with the tissues of at least one non-invasive electrode (18) has an approximately rectangular shape.
   37. The device according to any one of paragraphs 1, 11 to 13, 25, 34, 35, characterized in that the surface in contact with the tissues of at least one non-invasive electrode (18) has a shape approximately resembling a horseshoe.
   38. The device according to any one of paragraphs 1, 11 to 13, 25, 34 to 37, characterized in that the surface of at least one non-invasive electrode (18) is flexible.
   39. The device according to any one of paragraphs 1, 11 to 13, 25, 34, 35, characterized in that a non-invasive electrode (18) has the shape of a tip flattened at its end.
   40. The device according to any one of paragraphs 1, 34 to 39, characterized in that the non-invasive electrodes form an integral part of an elastic sheath (94).
   41. The device according to any one of paragraphs 1, 11 to 13, 25, 34.35, characterized in that at least one non-invasive electrode (18) is wire-shaped.
   42. The device according to any one of paragraphs 1, 34, 35, 41, characterized in that the device comprises only 2 wire-shaped non-invasive electrodes (18), these electrodes following a different axis.
   43. The device according to any one of paragraphs 1, 11 to 13, 25, 34, 35, 38, 40, characterized in that the surface in contact with the tissues of at least one non-invasive electrode (18) has an irregular shape.
   44. The device according to any one of paragraphs 1, 11 to 13, 25, 34 to 43, characterized in that the non-invasive electrodes (18) are asymmetrical.
   45. The device according to any one of paragraphs 1, 11 to 13, 25, 34 to 44, characterized in that at least one non-invasive electrode is composed of several non-invasive electrodes electrically connected to one another.
   46. A method implemented in a device according to any one of the previous paragraphs, for improving the penetration of molecules of active ingredient in vivo into the cells of the tissues of a human or animal subject, this method comprising the following stages:—
at least one electrode (9) electrically connected to the first terminal of a pulse generator (21) and at least one electrode (9) electrically connected to the second terminal of the pulse generator (21) are put into contact with the tissues to be treated and the active ingredient (36) is injected into the zone of tissues to be treated;
the emission of electric pulses by the generator (21) is then triggered, the amplitude of the electric signals being calculated as a function of the distance between the electrodes and the nature of the tissues, so as to create an electric field (12) between the electrodes, this allowing the active ingredient to penetrate into the tissues and into the cells.
   47. The method according to paragraph 46, characterized in that the active ingredient is injected into a sealed cavity (78) which contains the tissues to be treated and containing a fluid.
   48. The method according to paragraph 48, characterized in that the active ingredient is injected into the synovial cavity (78).
   49. The method according to any one of paragraphs 46 to 48, characterized in that, before delivering the fields:
a set of invasive electrodes (11, 10) connected to the two terminals of the generator is successively pushed into the tissues to intermediate depths;
the active ingredient is injected into the tissue at successive depths using the said electrodes (11);
all the invasive electrodes are then pushed in to a predefined final depth before triggering the delivery of the fields.
   50. The method according to any one of paragraphs 46 or 49, characterized in that, before delivering the fields:
the invasive electrodes connected to the first terminal of the generator (11) are introduced to the same depth into the tissues along the same axis and in the centre of the zone containing the active ingredient (36); and

the invasive electrodes (10) connected to the second terminal of the generator are introduced into the tissues, along the same axes and to substantially to the same depth as the invasive electrodes connected to the first terminal of the generator (11), approximately bordering the zone of tissues containing the active ingredient, the electrodes being positioned at an approximately identical distance from the centre of the zone containing the active ingredient (36) and being distributed regularly around this centre.

51. The method according to any one of paragraphs 46 to 51, characterized in that the active ingredient (36) is injected using the electrodes connected to the first terminal of the generator (11).

52. The method according to any one of paragraphs 47 to 48, characterized in that, before delivering the fields:

at least one catheter electrode (75) is connected to a terminal of a generator;
at least one non-invasive electrode (18) is connected to the other terminal of the generator;
each catheter electrode is pushed into the tissues in order to penetrate into the cavity; the needle (71) is then slid inside each catheter so as not to damage the walls of the cavity (77) during the shifting of the catheter electrode inside the cavity and during the delivery of the fields;
the active ingredient is injected using at least one catheter electrode; and

each non-invasive electrode is placed at the edge of the cavity.

53. The method according to any one of paragraphs 47 to 51, characterized in that at least one catheter electrode (75) is connected to one terminal of a generator, at least one catheter electrode (75) is connected to the other terminal of the generator and, before delivering the fields:
each catheter with electrodes is pushed into the tissues in order to penetrate into the cavity (78); the needle of each catheter is then slid so as not to damage the walls of the cavity during the shifting of the catheter electrode inside the cavity and during the delivery of the fields;
the catheter electrodes (75) being pushed in so as to be approximately parallel; and

active ingredient is injected using at least one catheter electrode.

54. The method according to paragraph 53, characterized in that at least one non-invasive electrode (18) is also connected to the terminals of the generator and placed on the tissues bordering the cavity (51).

55. The method according to one of paragraphs 52 or 54, characterized in that the non-invasive electrode is pressed against the tissues in order to get close to each catheter electrode.

56. The method according to any one of paragraphs 52 to 55, characterized in that at least one of the electrodes is a conductive catheter electrode used to inject the active ingredient having previously removed the needle from the catheter.

57. The method according to any one of paragraphs 52 to 56, characterized in that at least one needle (70) of a catheter-electrode is used to inject the active ingredient.

58. The method according to any one of paragraphs 52 to 57, characterized in that at least one of the electrodes is a conductive catheter electrode and in that the needle (71) is fully withdrawn from its respective catheter (70) before the delivery of the fields.

59. The method according to any one of paragraphs 52 to 58, characterized in that the active ingredient is injected continuously as the electrodes are pushed in.

60. The method according to any one of paragraphs 52 to 59, characterized in that the active ingredient is injected in successive stages as the electrodes are pushed in.

61. The method according to any one of paragraphs 46 to 60, characterized in that at least one invasive electrode (10, 11, 75) is gripped using a casing thus allowing it to be electrically connected to its terminal of the generator (21) and allowing a good grip on the electrode.

62. The method according to any one of paragraphs 53 to 61, characterized in that, once the catheter electrodes are introduced into the cavity (78), a means is used to modify the angle of the axis (69) of the electrodes in order to obtain a good parallelism and prevent any risk of contact between electrodes connected to different terminals of the generator.

63. The method according to paragraph 62, characterized in that, once the catheter electrodes are introduced into the cavity (78), a physical device is applied to the end (76) of the electrodes not penetrating into the tissues in order to modify their respective angle and make them parallel.

64. The method according to any one of paragraphs 50 to 63, characterized in that a means is used to ascertain the distance between the ends of the electrodes (74), and the relative position of the electrodes is adapted in order to obtain the electric field or electric current desired according to the targeted tissues.

65. The method according to any one of paragraphs 53 to 63, characterized in that a means is used to ascertain the distance between the ends of the electrodes (74), and the programming of the generator (21) is adapted in order to apply the voltage allowing the desired electric field or electric current to be obtained according to the targeted tissues.

66. The method according to any one of paragraphs 53 to 63, characterized in that the generator has a means of continuously ascertaining the distance between the ends of the electrodes (74), and can dynamically modify the programming of the pulses in order to apply the parameters allowing the desired electric field or electric current to be obtained according to the targeted tissues.

67. The method according to any one of paragraphs 46 to 52, 54 to 61, characterized in that the invasive electrodes (10, 11, 75) are parallel, made integral and held in place using a casing allowing a good grip on the device and allowing the electrodes to be connected to their respective generator terminal (21).

68. The method according to any one of paragraphs 46 to 48, characterized in that the first group of electrodes is composed of an invasive electrode, and that at least one electrode of the second group of electrodes is non-invasive (18) and is positioned on the surface of the tissues containing the ingredient.

69. The method according to paragraph 69, characterized in that at least one invasive electrode (11) of the first group of electrodes is pushed through organs in order to reach the zone of tissues containing the active ingredient without passing through the non-invasive electrode (18).

70. The method according to paragraph 69, characterized in that the invasive electrodes (11) are pushed in along an axis (69) forming part of a plane substantially parallel to the plane occupied by the surface of the plurality of non-invasive electrodes in contact with the zone of tissues containing the active ingredient.

71. The method according to any one of paragraphs 46 to 70, characterized in that the upper part of at least one invasive electrode is electrically insulated in order to prevent the passage of stray electric current (120) into the volume of tissues situated on the one hand between the electrodes and situated on the other hand between the surface of the skin (51) and the zone of tissues containing the active ingredient (36).

72. The method according to any one of paragraphs 46 to 11, characterized in that the upper part of all invasive electrodes is electrically insulated in order to prevent the passage of stray electric current (120) into the volume of tissues situated on the one hand between the electrodes and situated on the other hand between the surface of the skin (51) and the zone of tissues containing the active ingredient (36).

73. The method according to any one of paragraphs 46 to 48, characterized in that there are non-invasive electrodes of the first group of electrodes and the second set of electrodes on the surface of the tissues covering the zone containing the active ingredient, the electrodes being applied to a single face of the tissues, so as to permit the delivery of fields (121) spreading under the surface of the tissues (51) to which the electrodes are applied.

74. The method according to paragraph 73, characterized in that a set of non-invasive electrodes (18) is pressed against the surface of the tissues containing the active ingredient (51) in order to modify their geometry in order to increase the volume of tissues located between the electrodes.

75. The method according to any one of paragraphs 46 to 74, characterized in that the generator (21) is programmed to emit alternatively sequences of electric pulses between each pair of close electrodes:

in order to obtain, between each pulse (131) emitted by the generator in the zone where the tissues containing the active ingredient are located, an interval of less than 50 ms;
while still having, between two pulses (131) at a unitary electrode pair, an interval greater than 100 ms;
in order to reduce the harmfulness of the electric pulses.
   76. The method according to any one of paragraphs 46 to 75, characterized in that the duration (131) between each electric pulse emitted by the generator (21) towards a pair of electrodes is comprised between 1 ms and 50 ms, in order to reduce the harmfulness and reduce the intensity of the muscular contractions.
   77. The method according to any one of paragraphs 46 to 75, characterized in that the electric pulses emitted by the generator (21) are unipolar and have a square shape.
   78. The method according to any one of paragraphs 46 to 77, characterized in that:
the ratio between the potential difference between each electrode and their distance is comprised between 10 volt/cm and 750 volt/cm,
the duration of the electric pulses (132) is comprised between 1 and 250 ms,
the duration between the electric pulses (131) is comprised between 1 and 1500 ms, and
the number of pulses of each sequence of pulses (133) is comprised between 1 and 1000.
   79. The method according to any one of paragraphs 46 to 78, characterized in that the volt/cm ratio applied between the electrodes (10, 11, 18, 75) assumes a value comprised between 1.05 and 1.50 times the optimum value of the fields for the targeted tissues in order to increase the volume of tissues containing the active ingredient passed through by fields at an optimum number of volt/cm.
   80. The method according to any one of paragraphs 46 to 79, characterized in that a tranquillizer is injected before triggering the pulses using the generator (21).
   81. The method according to paragraph 80, characterized in that the tranquillizer used is xylazine.

1. A device for improving the in vivo penetration of molecules of active ingredient into the cells of the tissues of a human or animal subject, characterized in that it comprises:
a generator of electric pulses (21)
a first group of electrodes composed of at least one electrode (9) electrically connected to a first terminal of the generator of electric pulses,
a second group of electrodes composed of at least one electrode (9) electrically connected to the second terminal of the generator of electric pulses, and
a means (8) of injecting the active ingredient into the tissues.

2. The device according to claim 1, characterized in that the first group of electrodes is composed of a single electrode (11).

3. The device according to claim 2, characterized in that the electrode (11) is invasive.

4. The device according to claim 3, characterized in that the electrode (11) has a means of injecting the active ingredient.

5. The device according to claim 4, characterized in that the electrode (11) is an injection needle (8).

6. The device according to claim 5, characterized in that a means of injecting the active ingredient is constituted by at least one integral needle (8) and placed close to the electrode and at a depth less than the depth of the electrode.

7. The device according to claim 5, characterized in that:
the electrode is moreover constituted by a catheter envelope (70) pierced by orifices (78) of sufficient size to permit a contact between the needle and the tissues through these orifices;
the invasive needle of the electrode (71) passes through the catheter along its axis and can slide along the axis of the catheter and can be withdrawn from the catheter and,
the catheter and the needle, once assembled, form an invasive electrode, these electrodes being hereinafter called “electrode partly covered by a non-conductive catheter” (75).

8. The device according to any one of claims 4 to 6, characterized in that the electrode (11) is constituted by:
a catheter (70), covered by an electrically conductive surface (72) connected to the corresponding terminal of the pulse generator, hereinafter called “conductive catheter electrode”, its surface being composed of a material approximately retaining the flexibility of the catheter, which catheter can serve as means of injecting the active ingredient; and
an invasive needle (71) passing through the catheter along its axis and allowing penetration of the tissues and able to slide along the axis of the catheter and able to be withdrawn from the catheter, which needle can also serve as injection means.

9. The device according to any one of claims 3 to 8, characterized in that the invasive electrode is covered, in its upper part penetrating into the tissues, by an electric insulating material (15).

10. The device according to any one of claims 7 to 9, characterized in that at least one catheter electrode is magnetized (75).

11. The device according to any one of claims 3 to 10, characterized in that the second group of electrodes comprises at least one non-invasive electrode (18) arranged on the surface of the tissues covering the zone containing the active ingredient.

12. The device according to claim 11, characterized in that at least one non-invasive electrode (18) has an orifice (43) allowing the invasive electrode (11) to pass through it.

13. The device according to claim 12, characterized in that it also comprises a guide (49) allowing the axis of the invasive electrode (11) to be directed in a predefined direction.

14. The device according to any one of claims 3 to 10, characterized in that the second group of electrodes comprises a single invasive electrode (10).

15. The device according to claim 14, characterized in that the electrode of the second group is an electrode partly covered by a non-conductive catheter (75).

16. The device according to claim 14, characterized in that the electrode of the second group is constituted by a conductive catheter electrode (75).

17. The device according to one of claims 14 or 15, characterized in that the electrode of the first group is a catheter electrode (75) and, where the two needles are parallel, integral and connected by a support.

18. The device according to any one of claims 15 to 16, characterized in that the two invasive electrodes (10,11) are integral, assembled using a support (41), approximately parallel and approximately of the same depth.

19. The device according to any one of claims 4 to 10, characterized in that the second group of electrodes comprises a plurality of invasive electrodes (10).

20. The device according to claim 19, characterized in that the invasive electrodes of the second group (10) are situated approximately on a circle of which the electrode (11) of the first group approximately forms the centre.

21. The device according to one of claims 19 or 20, characterized in that the invasive electrodes of the second group border the zone where the active ingredient is injected (36).

22. The device according to any one of claims 19 to 21, characterized in that two invasive electrodes (100, 101) of the second group of electrodes are substantially aligned with the electrode of the first group (11).

23. The device according to any one of claims 14 to 16, 19 to 22, characterized in that it also comprises a means (764, 763) allowing orientation of the electrodes along a parallel axis.

24. The device according to any one of claims 20 to 22, characterized in that all the electrodes (10, 11) are integral, assembled using a support (41), approximately parallel and approximately of the same depth.

25. The device according to any one of claims 14 to 24, characterized in that the device also comprises at least one non-invasive electrode connected to one of the terminals of the generator.

26. The device according to any one of claims 4 to 16, 19 to 23 characterized in that:
each invasive electrode can have a casing (2) allowing a good grip on the electrode and providing the electric connection between the electrodes and its terminal of the generator (21); and
the casing (2) having a housing allowing the electrodes and the means of injecting the active ingredient and the reservoir (1) containing the active ingredient to be accommodated.

27. The device according to any one of claims 4 to 16, 19 to 23, characterized in that:
a plurality of electrodes have a single casing (2) allowing a good grip on the electrode device and providing the electric connection between each electrode and its terminal of the respective generator (21);
the casing (2) has a housing allowing the electrodes and the means of injecting the active ingredient and the reservoir (1) containing the active ingredient to be accommodated.

28. The device according to claim 23 or 27, characterized in that the casing also comprises the means of orienting the electrodes along a parallel axis.

29. The device according to one of claims 18 or 24, characterized in that:
the integral electrodes and their support have a casing (2) allowing a good grip on the electrode device and providing the electric connection between each electrode and its terminal of the generator (21); and
the casing (2) has a housing allowing the integral electrodes and their support and the means of injecting the active ingredient and the reservoir (1) containing the active ingredient to be accommodated.

30. The device according to one of claims 26 to 29, characterized in that the casing:
has a means allowing the electrode (11) to be successively pushed into the tissues to predefined intermediate depths;
and has a means allowing the injection of the active ingredient at each stop position.

31. The device according to claim 30, characterized in that the means allowing the electrode (11) to be successively pushed into the tissues to predefined intermediate depths is constituted by at least one stop (20).

32. The device according to any one of claims 2 to 31, characterized in that a plurality of invasive electrodes connected to the generator are covered, in their upper part penetrating into the tissues, by an electric insulating material (15).

33. The device according to any one of claims 2 to 32, characterized in that all the invasive electrodes connected to the generator are covered, in their upper part penetrating into the tissues, by an electric insulating material (15).

34. The device according to claim 1, characterized in that the two groups of electrodes comprise a set of non-invasive electrodes (18) arranged on the surface of the tissues covering the zone containing the active ingredient.

35. The device according to the claim 34, characterized in that the electrodes are applied to a single face of the tissues (51) containing the active ingredient so as permit the delivery of fields spreading under the surface of the tissues to which the electrodes are applied.

36. The device according to any one of claims 1, 11 to 13, 25, 34, 35, characterized in that the surface in contact with the tissues of at least one non-invasive electrode (18) has an approximately rectangular shape.

37. The device according to any one of claims 1, 11 to 13, 25, 34, 35, characterized in that the surface in contact with the tissues of at least one non-invasive electrode (18) has a shape approximately resembling a horseshoe.

38. The device according to any one of claims 1, 11 to 13, 25, 34 to 37, characterized in that the surface of at least one non-invasive electrode (18) is flexible.

39. The device according to any one of claims 1, 11 to 13, 25, 34, 35, characterized in that a non-invasive electrode (18) has the shape of a tip flattened at its end.

40. The device according to any one of claims 1, 34 to 39, characterized in that the non-invasive electrodes form an integral part of an elastic sheath (94).

41. The device according to any one of claims 1, 11 to 13, 25, 34.35, characterized in that at least one non-invasive electrode (18) is wire-shaped.

42. The device according to any one of claims 1, 34, 35, 41, characterized in that the device comprises only 2 wire-shaped non-invasive electrodes (18), these electrodes following a different axis.

43. The device according to any one of claims 1, 11 to 13, 25, 34, 35, 38, 40, characterized in that the surface in contact with the tissues of at least one non-invasive electrode (18) has an irregular shape.

44. The device according to any one of claims 1, 11 to 13, 25, 34 to 43, characterized in that the non-invasive electrodes (18) are asymmetrical.

45. The device according to any one of claims 1, 11 to 13, 25, 34 to 44, characterized in that at least one non-invasive electrode is composed of several non-invasive electrodes electrically connected to one another.

46. A method implemented in a device according to any one of the previous claims, for improving the penetration of molecules of active ingredient in vivo into the cells of the tissues of a human or animal subject, this method comprising the following stages:—
at least one electrode (9) electrically connected to the first terminal of a pulse generator (21) and at least one electrode (9) electrically connected to the second terminal of the pulse generator (21) are put into contact with the tissues to be treated and the active ingredient (36) is injected into the zone of tissues to be treated;
the emission of electric pulses by the generator (21) is then triggered, the amplitude of the electric signals being calculated as a function of the distance between the electrodes and the nature of the tissues, so as to create an electric field (12) between the electrodes, this allowing the active ingredient to penetrate into the tissues and into the cells.

47. The method according to claim 46, characterized in that the active ingredient is injected into a sealed cavity (78) which contains the tissues to be treated and containing a fluid.

48. The method according to claim 48, characterized in that the active ingredient is injected into the synovial cavity (78).

49. The method according to any one of claims 46 to 48, characterized in that, before delivering the fields:
a set of invasive electrodes (11, 10) connected to the two terminals of the generator is successively pushed into the tissues to intermediate depths;
the active ingredient is injected into the tissue at successive depths using the said electrodes (11);
all the invasive electrodes are then pushed in to a predefined final depth before triggering the delivery of the fields.

50. The method according to any one of claims 46 or 49, characterized in that, before delivering the fields:
the invasive electrodes connected to the first terminal of the generator (11) are introduced to the same depth into the tissues along the same axis and in the centre of the zone containing the active ingredient (36); and
the invasive electrodes (10) connected to the second terminal of the generator are introduced into the tissues, along the same axes and to substantially to the same depth as the invasive electrodes connected to the first terminal of the generator (11), approximately bordering the zone of tissues containing the active ingredient, the electrodes being positioned at an approximately identical distance from the centre of the zone containing the active ingredient (36) and being distributed regularly around this centre.

51. The method according to any one of claims 46 to 51, characterized in that the active ingredient (36) is injected using the electrodes connected to the first terminal of the generator (11).

52. The method according to any one of claims 47 to 48, characterized in that, before delivering the fields:
at least one catheter electrode (75) is connected to a terminal of a generator;
at least one non-invasive electrode (18) is connected to the other terminal of the generator;
each catheter electrode is pushed into the tissues in order to penetrate into the cavity; the needle (71) is then slid inside each catheter so as not to damage the walls of the cavity (77) during the shifting of the catheter electrode inside the cavity and during the delivery of the fields;
the active ingredient is injected using at least one catheter electrode; and
each non-invasive electrode is placed at the edge of the cavity.

53. The method according to any one of claims 47 to 51, characterized in that at least one catheter electrode (75) is connected to one terminal of a generator, at least one catheter electrode (75) is connected to the other terminal of the generator and, before delivering the fields:
each catheter with electrodes is pushed into the tissues in order to penetrate into the cavity (78); the needle of each catheter is then slid so as not to damage the walls of the cavity during the shifting of the catheter electrode inside the cavity and during the delivery of the fields;
the catheter electrodes (75) being pushed in so as to be approximately parallel; and
active ingredient is injected using at least one catheter electrode.

54. The method according to claim 53, characterized in that at least one non-invasive electrode (18) is also connected to the terminals of the generator and placed on the tissues bordering the cavity (51).

55. The method according to one of claims 52 or 54, characterized in that the non-invasive electrode is pressed against the tissues in order to get close to each catheter electrode.

56. The method according to any one of claims 52 to 55, characterized in that at least one of the electrodes is a conductive catheter electrode used to inject the active ingredient having previously removed the needle from the catheter.

57. The method according to any one of claims 52 to 56, characterized in that at least one needle (70) of a catheter-electrode is used to inject the active ingredient.

58. The method according to any one of claims 52 to 57, characterized in that at least one of the electrodes is a conductive catheter electrode and in that the needle (71) is fully withdrawn from its respective catheter (70) before the delivery of the fields.

59. The method according to any one of claims 52 to 58, characterized in that the active ingredient is injected continuously as the electrodes are pushed in.

60. The method according to any one of claims 52 to 59, characterized in that the active ingredient is injected in successive stages as the electrodes are pushed in.

61. The method according to any one of claims 46 to 60, characterized in that at least one invasive electrode (10, 11, 75) is gripped using a casing thus allowing it to be electrically connected to its terminal of the generator (21) and allowing a good grip on the electrode.

62. The method according to any one of claims 53 to 61, characterized in that, once the catheter electrodes are introduced into the cavity (78), a means is used to modify the angle of the axis (69) of the electrodes in order to obtain a good parallelism and prevent any risk of contact between electrodes connected to different terminals of the generator.

63. The method according to claim 62, characterized in that, once the catheter electrodes are introduced into the cavity (78), a physical device is applied to the end (76) of the electrodes not penetrating into the tissues in order to modify their respective angle and make them parallel.

64. The method according to any one of claims 50 to 63, characterized in that a means is used to ascertain the distance between the ends of the electrodes (74), and the relative position of the electrodes is adapted in order to obtain the electric field or electric current desired according to the targeted tissues.

65. The method according to any one of claims 53 to 63, characterized in that a means is used to ascertain the distance between the ends of the electrodes (74), and the programming of the generator (21) is adapted in order to apply the voltage allowing the desired electric field or electric current to be obtained according to the targeted tissues.

66. The method according to any one of claims 53 to 63, characterized in that the generator has a means of continuously ascertaining the distance between the ends of the electrodes (74), and can dynamically modify the programming of the pulses in order to apply the parameters allowing the desired electric field or electric current to be obtained according to the targeted tissues.

67. The method according to any one of claims 46 to 52, 54 to 61, characterized in that the invasive electrodes (10, 11, 75) are parallel, made integral and held in place using a casing allowing a good grip on the device and allowing the electrodes to be connected to their respective generator terminal (21).

68. The method according to any one of claims 46 to 48, characterized in that the first group of electrodes is composed of an invasive electrode, and that at least one electrode of the second group of electrodes is non-invasive (18) and is positioned on the surface of the tissues containing the ingredient.

69. The method according to claim 69, characterized in that at least one invasive electrode (11) of the first group of electrodes is pushed through organs in order to reach the zone of tissues containing the active ingredient without passing through the non-invasive electrode (18).

70. The method according to claim 69, characterized in that the invasive electrodes (11) are pushed in along an axis (69) forming part of a plane substantially parallel to the plane occupied by the surface of the plurality of non-invasive electrodes in contact with the zone of tissues containing the active ingredient.

71. The method according to any one of claims 46 to 70, characterized in that the upper part of at least one invasive electrode is electrically insulated in order to prevent the passage of stray electric current (120) into the volume of tissues situated on the one hand between the electrodes and situated on the other hand between the surface of the skin (51) and the zone of tissues containing the active ingredient (36).

72. The method according to any one of claims 46 to 11, characterized in that the upper part of all invasive electrodes is electrically insulated in order to prevent the passage of stray electric current (120) into the volume of tissues situated on the one hand between the electrodes and situated on the other hand between the surface of the skin (51) and the zone of tissues containing the active ingredient (36).

73. The method according to any one of claims 46 to 48, characterized in that there are non-invasive electrodes of the first group of electrodes and the second set of electrodes on the surface of the tissues covering the zone containing the active ingredient, the electrodes being applied to a single face of the tissues, so as to permit the delivery of fields (121) spreading under the surface of the tissues (51) to which the electrodes are applied.

74. The method according to claim 73, characterized in that a set of non-invasive electrodes (18) is pressed against the surface of the tissues containing the active ingredient (51) in order to modify their geometry in order to increase the volume of tissues located between the electrodes.

75. The method according to any one of claims 46 to 74, characterized in that the generator (21) is programmed to emit alternatively sequences of electric pulses between each pair of close electrodes:
in order to obtain, between each pulse (131) emitted by the generator in the zone where the tissues containing the active ingredient are located, an interval of less than 50 ms;
while still having, between two pulses (131) at a unitary electrode pair, an interval greater than 100 ms;
in order to reduce the harmfulness of the electric pulses.

76. The method according to any one of claims 46 to 75, characterized in that the duration (131) between each electric pulse emitted by the generator (21) towards a pair of electrodes is comprised between 1 ms and 50 ms, in order to reduce the harmfulness and reduce the intensity of the muscular contractions.

77. The method according to any one of claims 46 to 75, characterized in that the electric pulses emitted by the generator (21) are unipolar and have a square shape.

78. The method according to any one of claims 46 to 77, characterized in that:
the ratio between the potential difference between each electrode and their distance is comprised between 10 volt/cm and 750 volt/cm,
the duration of the electric pulses (132) is comprised between 1 and 250 ms,
the duration between the electric pulses (131) is comprised between 1 and 1500 ms, and the number of pulses of each sequence of pulses (133) is comprised between 1 and 1000.

79. The method according to any one of claims 46 to 78, characterized in that the volt/cm ratio applied between the electrodes (10, 11, 18, 75) assumes a value comprised between 1.05 and 1.50 times the optimum value of the fields for the targeted tissues in order to increase the volume of tissues containing the active ingredient passed through by fields at an optimum number of volt/cm.

80. The method according to any one of claims 46 to 79, characterized in that a tranquillizer is injected before triggering the pulses using the generator (21).

81. The method according to claim 80, characterized in that the tranquillizer used is xylazine.
